(19) United States
(12) Patent Application Publication
(10) Pub. No.: US 2025/0101039 A1
(43) Pub. Date: Mar. 27, 2025

(54) RIFAMYCIN DERIVATIVES FOR THE TREATMENT OF DISEASES

(71) Applicant: BIOVERSYS AG, Basel (CH)

(72) Inventors: Kevin ANTRAYGUES, Loos (FR); Marilyne BOUROTTE, Perenchies (FR); Glenn E. DALE, Basel (CH); Olivier DEFERT, Lille (FR); Bruna GIOIA, Lille (FR); Marc GITZINGER, Basel (CH); Sergio LOCIURO, Basel (CH); Mathieu MAINGOT, Lille (FR); Vincent TREBOSC, Kembs-Loechlé (FR); Nicolas WILLAND, Lille (FR)

(21) Appl. No.: 18/833,285

(22) PCT Filed: Jan. 27, 2023
(86) PCT No.: PCT/EP2023/051991

§ 371 Date: Jul. 25, 2024

(30) Foreign Application Priority Data
Jan. 28, 2022 (EP) 22154022.2

(51) Int. Cl. C07D 498/22; A61K 31/4188; A61P 31/04

(52) U.S. Cl. CPC C07D 498/22; A61K 31/4188; A61P 31/04

(57) ABSTRACT

The present disclosure relates to compounds and pharmaceutical compositions comprising the same for the treatment, amelioration and/or prevention of disease. In some embodiments, the disease is a bacterial infection. In some embodiments the infection is caused by a bacteria belonging to Acinetobacter spp., Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii; preferably the bacterial infection is caused by S. aureus, A. baumannii, E. faecium, E. faecalis, S. epidermidis, S. pneumoniae, S. pyogenes, H. influenzae, M. abscessus, M. tuberculosis, and/or M. smegmatis; yet more preferably the bacterial infection is caused by one or more bacterium belonging to the genus Acinetobacter; yet more preferably A. baumannii; and even more preferably A. baumannii that carries one or more resistance mechanisms against rifamycins such as rifabutin and/or rifampicin.

The present disclosure relates to compounds and pharmaceutical compositions comprising the same for the treatment, amelioration and/or prevention of disease. In some embodiments, the disease is a bacterial infection. In some embodiments, the bacteria belong to the genus Acinetobacter spp., Clostridium spp., Enterococcus spp., Hemophilus spp., Legionella spp., Mycobacterium spp., Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii. In some embodiments the infection is caused by a bacterium of the genus Acinetobacter.

### RELATED ART

Rifamycins such as rifabutin are known antibiotics with activity against a broad spectrum of pathogens such as Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and Toxoplasma gondii (Kunin, Clin. Infect. Dis., 1996; Farr and Mandell, Med. Clin. North. Am., 1982; Thornsberry et al., Rev. Infect. Dis., 1983; Hoover et al., Diagn. Microbiol. Infect. Dis., 1993; Kerry et al., J. Antimicrob. Chemother., 1975).

Rifabutin has been recently shown to have potent in vitro and in vivo activity against Mycobacterium abscessus (Aziz et al., Antimicrob. Agents Chemother., 2017; Dick et al., Antimicrob. Agents Chemother., 2020) and Acinetobacter baumannii (Luna et al., Nat. Microbiol., 2020; Trebosc et al., Drug Discov. Today, 2021; Trebosc et al., J. Antimicrob. Chemother., 2020). However, pathogens such as A. baumannii can show reduced rifabutin sensitivity through mechanisms such as mutation of the RNA polymerase ß-subunit (RpoB) gene (Trebosc et al., J. Antimicrob. Chemother., 2020). Accordingly, there is a need for more effective rifamycins for the treatment of bacterial infections such as those caused by a bacteria of the genus Acinetobacter.

### SUMMARY OF THE INVENTION

In a first aspect, the invention provides a compound of Formula I or a pharmaceutically acceptable salt, tautomer, solvate, hydrate, enantiomer or diastereomer thereof:

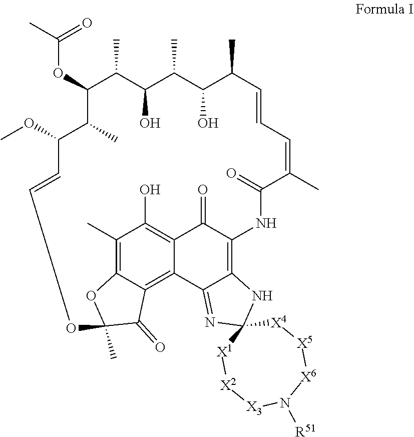

wherein:
X¹ is —CR¹R^{1A}—;
X² is independently, at each occurrence, selected from —CR²R^{2A}—, —NR^{2B}—, —O—, and a chemical bond;
X³ is independently, at each occurrence, selected from —CR³R^{3A}—, —NR^{3B}, —O—, and a chemical bond;
X⁴ is —CR⁴R^{4A}—;
X⁵ is independently, at each occurrence, selected from —CR⁵R^{5A}—, —NR^{5B}, —O—, and a chemical bond;
X⁶ is independently, at each occurrence, selected from —CR⁶R^{6A}—, —NR^{6B}—, —O—, and a chemical bond;
R¹ is independently, at each occurrence, selected from —H, —C₁-C₆ alkyl, and —C₃-C₈ cycloalkyl;
R¹, R^{1A}, R², R^{2A}, R³, R^{3A}, R⁴, R^{4A}, R⁵, R^{5A}, R⁶ and R^{6A} are independently, at each occurrence, selected from —H, —C₁-C₆ alkyl, and —C₁-C₆ alkoxy;
R^{2B}, R^{3B}, R^{5B} and R^{6B} are independently, at each occurrence, selected from —H and —C₁-C₆alkyl; or
any of R¹, R^{1A}, R², R^{2A}, R^{2B}, R³, R^{3A}, R^{3B}, R⁴, R^{4A}, R⁵, R^{5A}, R^{5B}, R⁶, R^{6A}, R^{6B} and R⁵¹, together with the atom to which they are attached, can independently combine with any other member of R¹, R^{1A}, R², R^{2A}, R^{2B}, R³, R^{3A}, R^{3B}, R⁴, R^{4A}, R⁵, R^{5A}, R^{5B}, R⁶, R^{6A}, R^{6B} and R⁵¹ to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₆ alkyl or —C₁-C₆ alkoxy;
wherein when any of R¹, R^{1A}, R², R^{2A}, R^{2B}, R³, R^{5A}, R^{3B}, R⁴, R^{4A}, R⁵, R^{5A}, R^{5B}, R⁶, R^{6A} and R^{6B} combine to form said first bridging cycloalkyl ring or said first bridging heterocycloalkyl ring, said first bridging cycloalkyl or first bridging heterocycloalkyl can independently combine with R⁵¹ to form a second bridging 5-7 membered heterocycloalkyl, wherein said second bridging heterocycloalkyl is optionally substituted by one or more —C₁-C₆ alkyl, or —C₁-C₆ alkoxy; and
provided that at least two of R¹, R^{1A}, R², R^{2A}, R^{2B}, R³, R^{3A}, R^{3B}, R⁴, R^{4A}, R⁵, R^{5A}, R^{5B}, R⁶, R^{6A}, R^{6B} and R⁵¹ combine to form a bridging cycloalkyl or heterocycloalkyl; or no more than one of X², X³, X⁵, and X⁶ is a chemical bond.

In one aspect, the invention provides a pharmaceutical composition comprising at least one compound as set forth herein, or a pharmaceutically acceptable salt, tautomer, solvate or hydrate thereof, and a pharmaceutically acceptable excipient.

In one aspect, the invention provides a compound or a pharmaceutical composition as described herein, for use in a method of preventing or treating a disease in a subject, preferably an infection, further preferably a bacterial infection, further preferably a bacterial infection caused by a bacteria belonging to Acinetobacter spp., Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii; more preferably a bacterial infection caused by S. aureus, A. baumannii, E. faecium, E. faecalis, S. epidermidis, S. pneumoniae, S. pyogenes, H. influenzae, M. abscessus, M. tuberculosis, and/or M. smegmatis; yet more preferably a bacterial infection caused by one or more bacterium belonging to the genus Acinetobacter; yet more preferably A. baumannii; and even more preferably A. baumannii that carries one or more resistance mechanisms against rifamycins such as rifabutin and/or rifampicin.

The inventive compounds are novel rifamycin derivatives containing novel modified spiro-imidazole moieties. In addition to showing broad antibacterial activity characteristic of the rifamycin class of antibiotics (e.g., against Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and Toxoplasma gondii), the inventive compounds additionally showed enhanced antibacterial activity against strains of the genus Acinetobacter. In particular, the inventive compounds exhibited activity against strains of A. baumannii that carry one or more resistance mechanisms against rifamycins such as rifampicin and/or rifabutin. Additional features and advantages of the inventive compounds are set forth in the Detailed Description, below.

### DESCRIPTION OF FIGURES

FIG. 1: ROESY and HSQC spectra superposition of Compound 13B.

FIG. 2: Attribution of the stereochemistry of the tropane moiety of Compound 13B.

### DETAILED DESCRIPTION OF THE INVENTION

The present disclosure provides novel rifamycin derivatives containing novel modified spiro-imidazole moieties that are effective at treating bacterial infections, preferably bacterial infections caused by a bacteria belonging to Acinetobacter spp., Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii; more preferably bacterial infections caused by S. aureus, A. baumannii, E. faecium, E. faecalis, S. epidermidis, S. pneumoniae, S. pyogenes, H. influenzae, M. abscessus, M. tuberculosis, and/or M. smegmatis; yet more preferably bacterial infections caused by one or more bacterium belonging to the genus Acinetobacter; yet more preferably A. baumannii; and even more preferably A. baumannii that carries one or more resistance mechanisms against rifamycins such as rifabutin and/or rifampicin.

The details of the technology are set forth in the accompanying description below. Although methods and materials similar or equivalent to those described herein can be used in the practice or testing of the present technology, illustrative methods and materials are now described. Other features, objects, and advantages of the disclosure will be apparent from the description and from the claims.

### Definitions

Unless defined otherwise, all technical and scientific terms used herein have the same meanings as commonly understood by one of ordinary skill in the art to which this invention belongs.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g. an alkyl group) can (but is not required to) be bonded other substituents (e.g. heteroatoms or alkoxy groups). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to, e.g., an alkoxy group or any other substituent described herein. Thus, the term “optionally substituted” means that a given chemical moiety has the potential to contain other functional groups, but does not necessarily have any further functional groups.

The term “alkyl” refers to a straight or branched chain saturated hydrocarbon. C₁-C₆ alkyl groups contain 1 to 6 carbon atoms. Examples of a C₁-C₆ alkyl group include, but are not limited to, methyl, ethyl, propyl, butyl, pentyl, isopropyl, isobutyl, sec-butyl and tert-butyl, isopentyl and neopentyl.

The terms “alkylene” or “alkylenyl,” as used herein, refer to a straight or branched hydrocarbon chain bi-radical derived from alkyl, as defined herein, wherein one hydrogen of said alkyl is cleaved off generating the second radical of said alkylene. Examples of alkylene are, by way of illustration, —CH₂—, —CH₂—CH₂—, —CH(CH₃)—, —CH₂—CH₂—CH₂—, —CH(CH₃)—CH₂—, or —CH(CH₂CH₃)—.

The term “cycloalkyl” means monocyclic saturated carbon rings containing 3-8 carbon atoms. A C₃-C₆ cycloalkyl contains between 3 and 6 carbon atoms. Examples of cycloalkyl groups include, without limitations, cyclopropyl, cyclobutyl, cyclopentyl, cyclohexyl, cycloheptanyl, and cyclooctanyl.

The terms “heterocyclyl” or “heterocycloalkyl” or “heterocycle” refer to 3 to 8-membered rings containing carbon and heteroatoms taken from oxygen, nitrogen, sulfur, and/or phosphorous (preferably oxygen and nitrogen) and wherein the ring does not comprise delocalized π electrons (aromaticity) shared among the ring carbon or heteroatoms. In preferred embodiments, the heterocycloalkyl ring is fully saturated. Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxanyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. In some embodiments, the heterocyclyl or heterocycloalkyl ring is bridged, e.g., forms a bicyclic or tricyclic ring with the atoms or substituents to which it is attached.

“C₁-C₆-alkoxy”, as used herein, refers to a substituted hydroxyl of the formula (—OR′), wherein R′ is an optionally substituted C₁-C₆ alkyl, as defined herein, and the oxygen moiety is directly attached to the parent molecule, and thus the term “C₁-C₆ alkoxy”, as used herein, refers to straight chain or branched C₁-C₆ alkoxy which may be, for example, methoxy, ethoxy, n-propoxy, isopropoxy, n-butoxy, isobutoxy, sec-butoxy, tert-butoxy, straight or branched pentoxy, straight or branched hexyloxy. Preferred are C₁-C₄ alkoxy and C₁-C₃ alkoxy.

The term “pharmaceutically acceptable salt” as used herein refers to a salt that possesses the desired pharmacological activity of the parent compound. Such salts include acid addition salts formed with inorganic acids or organic acids known to the skilled person in the art (P. Heinrich Stahl (Editor), Camille G. Wermuth (Editor); Handbook of Pharmaceutical Salts: Properties, Selection, and Use, 2nd Revised Edition, March 2011, Wiley-VCH, ISBN: 978-3-90639-051-2). Representative “pharmaceutically acceptable salts” of the inventive compounds include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, hydroiodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “stereoisomers” refers to the set of compounds which have the same number and type of atoms and share the same bond connectivity between those atoms, but differ in three-dimensional structure. The term “stereoisomer” refers to any member of this set of compounds.

The term “diastereomers” refers to the set of stereoisomers which cannot be made superimposable by rotation around single bonds. For example, cis- and trans-double bonds, endo- and exo-substitution on bicyclic ring systems, and compounds containing multiple stereogenic centers with different relative configurations are considered to be diastereomers. The term “diastereomer” refers to any member of this set of compounds. In some examples presented, the synthetic route may produce a single diastereomer or a mixture of diastereomers. In such cases all possible diastereomers are contemplated by the present invention.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers.

The term “tautomers” refers to a set of compounds that have the same number and type of atoms, but differ in bond connectivity and are in equilibrium with one another. A “tautomer” is a single member of this set of compounds. Typically a single tautomer is drawn but it is understood that this single structure is meant to represent all possible tautomers that might exist. Examples include enol-ketone tautomerism. When a ketone is drawn it is understood that both the enol and ketone forms are part of the present disclosure.

The term “solvate” refers to a complex of variable stoichiometry formed by a solute and solvent. Such solvents for the purpose of the disclosure do not interfere with the biological activity of the solute. Examples of suitable solvents include, but are not limited to, water, MeOH, EtOH, and AcOH. Solvates wherein water is the solvent molecule are typically referred to as “hydrates.” Hydrates include compositions containing stoichiometric amounts of water, as well as compositions containing variable amounts of water.

An “effective amount” when used in connection with a compound is an amount effective for treating or preventing a disease in a subject as described herein.

The term “carrier”, as used in this disclosure, encompasses excipients and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition comprising the same to a subject.

A “patient” or “subject” is a mammal, e.g., a human, mouse, rat, guinea pig, dog, cat, horse, cow, pig, or non-human primate, such as a monkey, chimpanzee, baboon or rhesus, preferably a human.

### Compounds of the Invention

In a first aspect, the invention provides a compound of Formula I or a pharmaceutically acceptable salt, tautomer, solvate, hydrate, enantiomer or diastereomer thereof:

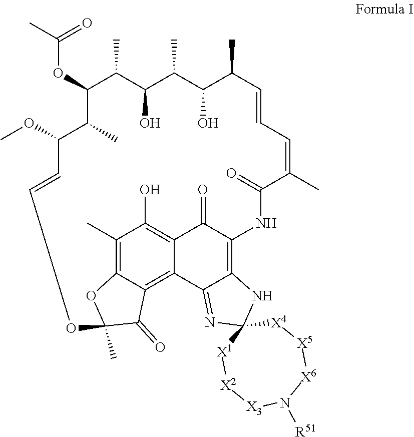

wherein:
X¹ is —CR¹R^{1A}—;
X² is independently, at each occurrence, selected from —CR²R^{2A}—, —NR^{2B}—, —O—, and a chemical bond;
X³ is independently, at each occurrence, selected from —CR³R^{3A}—, —NR^{3B}—, —O—, and a chemical bond;
X⁴ is —CR⁴R^{4A}—;
X⁵ is independently, at each occurrence, selected from —CR⁵R^{5A}—, —NR⁵¹—, —O—, and a chemical bond;
X⁶ is independently, at each occurrence, selected from —CR⁶R^{6A}—, —NR^{6B}—, —O—, and a chemical bond;
R⁵¹ is independently, at each occurrence, selected from —H, —C₁-C₆ alkyl, and —C₃-C₈ cycloalkyl;
R¹, R^{1A}, R², R^{2A}, R³, R^{3A}, R⁴, R^{4A}, R⁵, R^{5A}, R⁶ and R^{6A} are independently, at each occurrence, selected from —H, —C₁-C₆ alkyl, and —C₁-C₆ alkoxy;
R^{2B}, R^{3B}, R^{5B} and R^{6B} are independently, at each occurrence, selected from —H and —C₁-C₆ alkyl; or
any of R¹, R^{1A}, R², R^{2A}, R^{2B}, R³, R^{3A}, R^{3B}, R⁴, R^{4A}, R⁵, R^{5A}, R^{5B}, R⁶, R^{6A}, R^{6B} and R⁵¹, together with the atom to which they are attached, can independently combine with any other member of R¹, R^{1A}, R², R^{2A}, R^{2B}, R³, R^{3A}, R^{3B}, R⁴, R^{4A}, R⁵, R^{5A}, R^{5B}, R⁶, R^{6A}, R^{6B} and R⁵¹ to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₆ alkyl or —C₁-C₆ alkoxy;
wherein when any of R¹, R^{1A}, R², R^{2A}, R^{2B}, R³, R^{3A}, R^{3B}, R⁴, R^{4A}, R⁵, R^{5A}, R^{5B}, R⁶, R^{6A} and R^{6B} combine to form said first bridging cycloalkyl ring or said first bridging heterocycloalkyl ring, said first bridging cycloalkyl or first bridging heterocycloalkyl can independently combine with R⁵¹ to form a second bridging 5-7 membered heterocycloalkyl, wherein said second bridging heterocycloalkyl is optionally substituted by one or more —C₁-C₆ alkyl, or —C₁-C₆ alkoxy; and
provided that at least two of R¹, R^{1A}, R², R^{2A}, R^{2B}, R³, R^{3A}, R^{3B}, R⁴, R^{4A}, R⁵, R^{5A}, R^{5B}, R⁶, R^{6A}, R^{6B} and R⁵¹ combine to form a bridging cycloalkyl or heterocycloalkyl; or no more than one of X², X³, X⁵, and X⁶ is a chemical bond.

In some embodiments, X¹, X², X³, X⁴, X⁵, X⁶ and NR⁵¹ combine to form an azepane, wherein said azepane optionally further comprises a bridging C₄-C₈ cycloalkyl or bridging 5 to 8-membered heterocycloalkyl; or
X¹, X², X³, X⁴, X⁵, X⁶ and NR⁵¹ combine to form a piperidine further comprising a bridging C₄-C₈ cycloalkyl or bridging 5 to 8-membered heterocycloalkyl.

In some embodiments, X¹ is —CR¹R^{1A}—;
X² is independently, at each occurrence, selected from —CR²R^{2A}—, —NR^{2B}—, and —O—;
X³ is independently, at each occurrence, selected from —CR³R^{3A}—, —NR^{3B}—, —O—, and a chemical bond;
X⁴ is —CR⁴R^{4A}—;
X⁵ is independently, at each occurrence, selected from —CR⁵R^{5A}—, —NR^{5B}—, and —O—;
X⁶ is independently, at each occurrence, selected from —CR⁶R^{6A}—, —NR^{6B}, —O—, and a chemical bond;
R⁵¹ is independently, at each occurrence, selected from —H, —C₁-C₆ alkyl, and —C₃-C₈ cycloalkyl;
R¹, R^{1A}, R², R^{2A}, R³, R^{3A}, R⁴, R^{4A}, R⁵, R^{5A}, R⁶ and R^{6A} are independently, at each occurrence, selected from —H, —C₁-C₆ alkyl, and —C₁-C₆ alkoxy;
R^{2B}, R^{3B}, R^{5B} and R^{6B} are independently, at each occurrence, selected from —H and —C₁-C₆ alkyl; or
any of R¹, R², R³, R⁴, R⁵, R⁶, and R⁵¹, together with the atom to which they are attached, can independently combine with any other member of R¹, R², R³, R⁴, R⁵, R⁶, R^{6A}, and R⁵¹ to form a first bridging C₄-C₈ cycloalkyl ring or a bridging 5-8 membered heterocycloalkyl ring, wherein each bridging cycloalkyl or bridging heterocycloalkyl is optionally substituted by one or more —C₁-C₆ alkyl or —C₁-C₆ alkoxy;
wherein when any of R¹, R², R³, R⁴, R⁵, and R⁶ combine to form a first bridging cycloalkyl ring or a first bridging heterocycloalkyl ring, the first bridging cycloalkyl or first bridging heterocycloalkyl can independently combine with R⁵¹ to form a second bridging 5-7 membered heterocycloalkyl, wherein said second bridging heterocycloalkyl is optionally substituted by one or more —C₁-C₆ alkyl or —C₁-C₆ alkoxy; and
provided that at least two of R¹, R², R³, R⁴, R⁵, R⁶, and R⁵¹ combine to form a bridging cycloalkyl or heterocycloalkyl; or no more than one of X¹, X², X³, X⁴, X⁵, and X⁶ is a chemical bond.

In some embodiments, X¹ is CR¹R^{1A}; X² is CR²R^{2A}; X³ is independently, at each occurrence, selected from CR³R^{3A} and a chemical bond; X⁴ is CR⁴R^{4A}; X⁵ is CR⁵R^{5A}; X⁶ is independently, at each occurrence, selected from CR⁶R^{6A} and a chemical bond;
R¹, R^{1A}, R², R^{2A}, R³, R^{3A}, R⁴, R^{4A}, R⁵, R^{5A}, R⁶ and R^{6A} are independently, at each occurrence, selected from —H, —C₁-C₆ alkyl, and —C₁-C₆ alkoxy; R⁵¹ is independently, at each occurrence, selected from —H, —C₁-C₆ alkyl, and —C₃-C₈ cycloalkyl; or
any of R¹, R² and R³, together with the carbon atoms to which they are attached, can independently combine with any of R⁴, R⁵, R⁶, and R⁵¹ to form a bridging C₄-C₈ cycloalkyl ring or a bridging 5-8 membered heterocycloalkyl ring; or any of R⁴, R⁵, R⁶, together with the carbon atoms to which they are attached, can independently combine with R⁵¹ to form a bridging 5-8 membered heterocycloalkyl ring, wherein said heterocycloalkyl is optionally substituted by one or more —C₁-C₆ alkyl or —C₁-C₆ alkoxy;
wherein when any of R¹, R², R³, R⁴, R⁵, and R⁶, and combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted by one or more —C₁-C₆ alkyl, or —C₁-C₆ alkoxy; and
provided that at least two of R¹, R², R³, R⁴, R⁵, R⁶ and R⁵¹ combine to form a bridging cycloalkyl or heterocycloalkyl; or no more than one of X³ and X⁶ is a chemical bond.

In some embodiments, X¹ is CR¹R^{1A}; X² is CR²R^{2A}; X³ is independently, at each occurrence, selected from CR³R^{3A} and a chemical bond; X⁴ is CR⁴R^{4A}; X⁵ is CR⁵R^{5A}; X⁶ is independently, at each occurrence, selected from CR⁶R^{6A} and a chemical bond;
R¹, R^{1A}, R², R^{2A}, R³, R^{3A}, R⁴, R^{4A}, R⁵, R^{5A}, R⁶ and R^{6A} are independently, at each occurrence, selected from —H, —C₁-C₆ alkyl, and —C₁-C₆ alkoxy;
R⁵¹ is independently, at each occurrence, selected from —H, —C₁-C₆ alkyl, and —C₃-C₈ cycloalkyl; or
any of R¹, R² and R³, together with the carbon atoms to which they are attached, can independently combine with any of R⁴, R⁵, R⁶, and R⁵¹ to form a bridging C₄-C₈ cycloalkyl ring or a bridging 5-8 membered heterocycloalkyl ring; or any of R⁴, R⁵, R⁶, together with the carbon atoms to which they are attached, can independently combine with R⁵¹ to form a bridging C₄-C₈ cycloalkyl ring or a bridging 5-8 membered heterocycloalkyl ring, wherein each bridging cycloalkyl or bridging heterocycloalkyl is optionally substituted by one or more —C₁-C₆ alkyl or —C₁-C₆ alkoxy;
wherein when any of R¹, R², and R³ combine with any of R⁴, R⁵, and R⁶ to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted by one or more —C₁-C₆ alkyl, or —C₁-C₆ alkoxy; and
provided that at least two of R¹, R², R³, R⁴, R⁵, R⁶, and R⁵¹ combine to form a bridging cycloalkyl or heterocycloalkyl; or no more than one of X³ and X⁶ is a chemical bond.

In some embodiments, X¹ is CR¹R^{1A}; X² is CR²R^{2A}; X³ is independently, at each occurrence, selected from CR³R^{3A} and a chemical bond; X⁴ is CR⁴R^{4A}; X⁵ is CR⁵R^{5A}; X⁶ is independently, at each occurrence, selected from CR⁶R^{6A} and a chemical bond;
R¹, R^{1A}, R², R^{2A}, R³, R^{3A}, R⁴, R^{4A}, R⁵, R^{5A}, R⁶ and R^{6A} are independently, at each occurrence, selected from —H, —C₁-C₄ alkyl, and —C₁-C₄ alkoxy, preferably —H, C₁-C₃ alkyl and C₁-C₃ alkoxy, more preferably —H, C₁-C₂ alkyl and —C₂ alkoxy, yet more preferably —H;
R⁵¹ is independently, at each occurrence, selected from —H, —C₁-C₄ alkyl, and —C₃-C₆ cycloalkyl; or
any of R¹, R² and R³, together with the carbon atoms to which they are attached, can independently combine with any of R⁴, R⁵, R⁶, and R⁵¹ to form a bridging C₄-C₈ cycloalkyl ring or a bridging 5-8 membered heterocycloalkyl ring; or any of R⁴, R⁵, R⁶, together with the carbon atoms to which they are attached, can independently combine with R⁵¹ to form a bridging C₄-C₈ cycloalkyl ring or a bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably one or more —C₁-C₃ alkyl or —C₁-C₃alkoxy, more preferably one or more —C₁-C₃ alkyl or —C₁-C₃alkoxy;
wherein when any of R¹, R², and R³ combine any of R⁴, R⁵, and R⁶ to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted by one or more —C₁-C₄ alkyl or —C₁-C₄ alkoxy, preferably —C₁-C₃ alkyl, or —C₁-C₃ alkoxy, more preferably —C₁-C₂ alkyl, or C₁-C₂ alkoxy;
provided that (i) R¹ and R⁴ combine to form a first bridging cycloalkyl or heterocycloalkyl and X³ and X⁶ are chemical bonds; (ii) R² and R⁵ combine to form a first bridging cycloalkyl or heterocycloalkyl and X³ and X⁶ are chemical bonds; (iii) one of R¹ and R⁴ combine with R⁵¹ to form a first bridging cycloalkyl ring or first bridging heterocycloalkyl ring and X³ and X⁶ are chemical bonds; or (iv) exactly one of X³ and X⁶ is a chemical bond.

In some embodiments, X¹ is CR¹R^{1A}; X² is CR²R^{2A}; X³ is independently, at each occurrence, selected from CR³R^{3A} and a chemical bond; X⁴ is CR⁴R^{4A}; X⁵ is CR⁵R^{5A}; X⁶ is independently, at each occurrence, selected from CR⁶R^{6A} and a chemical bond;
R¹, R^{1A}, R², R^{2A}, R³, R^{3A}, R⁴, R^{4A}, R⁵, R^{5A}, R⁶ and R^{6A} are independently, at each occurrence, selected from —H, —C₁-C₄ alkyl, and —C₁-C₄ alkoxy, preferably —H, C₁-C₃ alkyl and C₁-C₃ alkoxy, more preferably —H, C₁-C₂ alkyl and —C₂ alkoxy, yet more preferably —H;
R⁵¹ is independently, at each occurrence, selected from —H, —C₁-C₄ alkyl, and —C₃-C₆ cycloalkyl; or
any of R¹, R² and R³, together with the carbon atoms to which they are attached, can independently combine with any of R⁴, R⁵, R⁶, and R⁵¹ to form a bridging C₄-C₈ cycloalkyl ring or a bridging 5-8 membered heterocycloalkyl ring; or any of R⁴, R⁵, R⁶, together with the carbon atoms to which they are attached, can independently combine with R⁵¹ to form a bridging C₄-C₈ cycloalkyl ring or a bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably one or more —C₁-C₃ alkyl or —C₁-C₃alkoxy, more preferably one or more —C₁-C₃ alkyl or —C₁-C₃alkoxy;
wherein when any of R¹, R², and R³ combine any of R⁴, R⁵, and R⁶ to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted by one or more —C₁-C₄ alkyl or —C₁-C₄ alkoxy, preferably —C₁-C₃ alkyl, or —C₁-C₃ alkoxy, more preferably —C₁-C₂ alkyl, or C₁-C₂ alkoxy;
provided that (i) R¹ and R⁴ combine to form a bridging cycloalkyl or heterocycloalkyl and X³ and X⁶ are chemical bonds; (ii) R² and R⁵ combine to form a bridging cycloalkyl or heterocycloalkyl and X³ and X⁶ are chemical bonds; or (iii) exactly one of X³ and X⁶ is a chemical bond.

In some embodiments, X¹ is CR¹R^{1A}; X² is CR²R^{2A}; X³ is independently, at each occurrence, selected from CR³R^{3A} and a chemical bond; X⁴ is CR⁴R^{4A}; X⁵ is CR⁵R^{5A}; X⁶ is independently, at each occurrence, selected from CR⁶R^{6A} and a chemical bond; wherein
(i) R^{1A}, R², R^{2A}, R^{4A}, R⁵ and R^{5A} are —H; R³, R^{3A}, R⁶ and R^{6A} are absent or —H; R¹ and R⁴ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(ii) R^{1A}, R¹, R^{2A}, R^{4A}, R⁴ and R^{5A} are —H; R³, R^{3A}, R⁶ and R^{6A} are absent or —H; R² and R⁵ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(iii) R^{1A}, R², R^{2A}, R⁴, R^{4A}, R⁵ and R^{5A} are —H; R³, R^{3A}, R⁶ and R^{6A} are absent or —H; R¹ and R⁵¹ combine to form a bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(iv) R¹, R^{1A}, R², R^{2A}, R^{4A}, R⁵, and R^{5A} are —H; R³, R^{3A}, R⁶ and R^{6A} are absent or —H; R⁴ and R⁵¹ combine to form a bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; wherein
when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted with one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(v) exactly one of X³ and X⁶ is a chemical bond, optionally wherein any of R¹, R², R⁴, R⁵ and the remaining R³ or R⁶ can combine to form a bridging C₄-C₈ cycloalkyl or bridging 5 to 8-membered heterocycloalkyl.

In some embodiments, X¹ is CR¹R^{1A}; X² is CR²R^{2A}; X³ is independently, at each occurrence, selected from CR³R^{3A} and a chemical bond; X⁴ is CR⁴R^{4A}; X⁵ is CR⁵R^{5A}; X⁶ is independently, at each occurrence, selected from CR⁶R^{6A} and a chemical bond; wherein
(i) R^{1A}, R², R^{2A}, R^{4A}, R⁵ and R^{5A} are —H; R³, R^{3A}, R⁶ and R^{6A} are absent or —H; R¹ and R⁴ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(ii) R^{1A}, R¹, R^{2A}, R^{4A}, R⁴ and R^{5A} are —H; R³, R^{3A}, R⁶ and R^{6A} are absent or —H; R² and R⁵ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(iii) R^{1A}, R², R^{2A}, R⁴, R^{4A}, R⁵ and R^{5A} are —H; R³, R^{3A}, R⁶ and R^{6A} are absent or —H; R¹ and R⁵¹ combine to form a bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(iv) R¹, R^{1A}, R², R^{2A}, R^{4A}, R⁵, and R^{5A} are —H; R³, R^{3A}, R⁶ and R^{6A} are absent or —H; R⁴ and R⁵¹ combine to form a bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; wherein
when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted with one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(v) exactly one of X³ and X⁶ is a chemical bond.

In some embodiments, X¹ is CR¹R^{1A}; X² is CR²R^{2A}; X³ is independently, at each occurrence, selected from CR³R^{3A} and a chemical bond, preferably X³ is a chemical bond and R³ and R^{3A} are absent; X⁴ is CR⁴R^{4A}; X⁵ is CR⁵R^{5A}; X⁶ is independently, at each occurrence, selected from CR⁶R^{6A} and a chemical bond, preferably X⁶ is a chemical bond and R⁶ and R^{6A} are absent; wherein
(i) R^{1A}, R², R^{2A}, R^{4A}, R⁵ and R^{5A} are —H; R³, R^{3A}, R⁶ and R^{6A} are absent or —H, preferably R³, R^{3A}, R⁶ and R^{6A} are absent; R¹ and R⁴ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(ii) R^{1A}, R¹, R^{2A}, R^{4A}, R⁴ and R^{5A} are —H; R³, R^{3A}, R⁶ and R^{6A} are absent or —H, preferably R³, R^{3A}, R⁶ and R^{6A} are absent; R² and R⁵ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(iii) R^{1A}, R², R^{2A}, R⁴, R^{4A}, R⁵ and R^{5A} are —H; R³, R^{3A}, R⁶ and R^{6A} are absent or —H, preferably R³, R^{3A}, R⁶ and R^{6A} are absent; R¹ and R⁵¹ combine to forma bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(iv) R¹, R^{1A}, R², R^{2A}, R^{4A}, R⁵, and R^{5A} are —H; R³, R^{3A}, R⁶ and R^{6A} are absent or —H, preferably R³, R^{3A}, R⁶ and R^{6A} are absent; R⁴ and R⁵¹ combine to form a bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; wherein when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted with one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(v) exactly one of X³ and X⁶ is a chemical bond, optionally wherein any of R¹, R², R⁴, R⁵ and the remaining R³ or R⁶ can combine to form a bridging C₄-C₈ cycloalkyl or bridging 5 to 8-membered heterocycloalkyl.

In some embodiments, X¹ is CR¹R^{1A}; X² is CR²R^{2A}; X³ is independently, at each occurrence, selected from CR³R^{3A} and a chemical bond, preferably X³ is a chemical bond and R³ and R^{3A} are absent; X⁴ is CR⁴R^{4A}; X⁵ is CR⁵R^{5A}; X⁶ is independently, at each occurrence, selected from CR⁶R^{6A} and a chemical bond, preferably X⁶ is a chemical bond and R⁶ and R^{6A} are absent; wherein
(i) R^{1A}, R², R^{2A}, R^{4A}, R⁵ and R^{5A} are —H; R³, R^{3A}, R⁶ and R^{6A} are absent or —H, preferably R³, R^{3A}, R⁶ and R^{6A} are absent; R¹ and R⁴ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(ii) R^{1A}, R¹, R^{2A}, R^{4A}, R⁴ and R^{5A} are —H; R³, R^{3A}, R⁶ and R^{6A} are absent or —H, preferably R³, R^{3A}, R⁶ and R^{6A} are absent; R² and R⁵ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy;
or
(iii) R^{1A}, R², R^{2A}, R⁴, R^{4A}, R⁵ and R^{5A} are —H; R³, R^{3A}, R⁶ and R^{6A} are absent or —H, preferably R³, R^{3A}, R⁶ and R^{6A} are absent; R¹ and R⁵¹ combine to forma bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(iv) R¹, R^{1A}, R², R^{2A}, R^{4A}, R⁵, and R^{5A} are —H; R³, R^{3A}, R⁶ and R^{6A} are absent or —H, preferably R³, R^{3A}, R⁶ and R^{6A} are absent; R⁴ and R⁵¹ combine to forma bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; wherein when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted with one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(v) exactly one of X³ and X⁶ is a chemical bond.

In some embodiments, X¹ is CR¹R^{1A}; X² is CR²R^{2A}; X⁴ is CR⁴R^{4A}; X⁵ is CR⁵R^{5A};
(i) X³ and X⁶ are both chemical bonds; R^{1A}, R², R^{2A}, R^{4A}, R⁵, and R^{5A} are —H; R¹ and R⁴ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₃ alkyl or —C₁-C₃ alkoxy, preferably —C₁-C₂ alkyl or —C₁-C₂ alkoxy; or
(ii) X³ and X⁶ are both chemical bonds; R^{1A}, R¹, R^{2A}, R^{4A}, R⁴, and R^{5A} are —H; R² and R⁵ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₃ alkyl or —C₁-C₃ alkoxy, preferably —C₁-C₂ alkyl or —C₁-C₂ alkoxy; or
(iii) X³ and X⁶ are both chemical bonds; R^{1A}, R², R^{2A}, R^{4A}, R⁵, and R^{5A} are —H; one of R¹ and R⁴ combine with R⁵¹ to form a bridging 5-8 membered heterocycloalkyl ring, wherein said bridging heterocycloalkyl is optionally substituted by one or more —C₁-C₃ alkyl or —C₁-C₃ alkoxy, preferably —C₁-C₂ alkyl or —C₁-C₂ alkoxy; wherein when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl wherein the second bridging heterocycloalkyl is optionally substituted by one or more —C₁-C₃ alkyl or —C₁-C₃ alkoxy, preferably —C₁-C₂ alkyl or —C₁-C₂ alkoxy; or
(iv) exactly one of X³ and X⁶ is a chemical bond, optionally wherein any of R¹, R², R⁴, R⁵ and the remaining R³ or R⁶ can combine to form a bridging C₄-C₈ cycloalkyl or bridging 5 to 8-membered heterocycloalkyl.

In some embodiments, X¹ is CR¹R^{1A}; X² is CR²R^{2A}; X⁴ is CR⁴R^{4A}; X⁵ is CR⁵R^{5A};
(i) X³ and X⁶ are both chemical bonds; R^{1A}, R², R^{2A}, R^{4A}, R⁵, and R^{5A} are —H; R¹ and R⁴ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₃ alkyl or —C₁-C₃ alkoxy, preferably —C₁-C₂ alkyl or —C₁-C₂ alkoxy; or
(ii) X³ and X⁶ are both chemical bonds; R^{1A}, R¹, R^{2A}, R^{4A}, R⁴, and R^{5A} are —H; R² and R⁵ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₃ alkyl or —C₁-C₃ alkoxy, preferably —C₁-C₂ alkyl or —C₁-C₂ alkoxy; or
(iii) X³ and X⁶ are both chemical bonds; R^{1A}, R², R^{2A}, R^{4A}, R⁵, and R^{5A} are —H; one of R¹ and R⁴ combine with R⁵¹ to form a bridging 5-8 membered heterocycloalkyl ring, wherein said bridging heterocycloalkyl is optionally substituted by one or more —C₁-C₃ alkyl or —C₁-C₃ alkoxy, preferably —C₁-C₂ alkyl or —C₁-C₂ alkoxy; wherein when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl wherein the second bridging heterocycloalkyl is optionally substituted by one or more —C₁-C₃ alkyl or —C₁-C₃ alkoxy, preferably —C₁-C₂ alkyl or —C₁-C₂ alkoxy; or
(iv) exactly one of X³ and X⁶ is a chemical bond.

In some embodiments, X¹ is CR¹R^{1A}; X² is CR²R^{2A}; X⁴ is CR⁴R^{4A}; X⁵ is CR⁵R^{5A};
(i) X³ and X⁶ are both chemical bonds; R^{1A}, R², R^{2A}, R^{4A}, R⁵, and R^{5A} are —H; R¹ and R⁴ combine to form a first bridging C₄-C₆ cycloalkyl ring or a first bridging 5-7 membered heterocycloalkyl ring, wherein the heterocycloalkyl ring comprises one or more atoms selected form O and N, preferably one or two atoms selected from O and N, more preferably exactly one atom selected from O and N, wherein each bridging cycloalkyl or bridging heterocycloalkyl is optionally substituted by one or more —C₁-C₃ alkyl or —C₁-C₃ alkoxy, more preferably —C₁-C₂ alkyl or —C₁-C₂ alkoxy; or
(ii) X³ and X⁶ are both chemical bonds; R^{1A}, R¹, R^{2A}, R^{4A}, R⁴, and R^{5A} are —H, R² and R⁵ combine to form a first bridging C₅-C₇ cycloalkyl ring or a first bridging 5-7 membered heterocycloalkyl ring, wherein the heterocycloalkyl ring comprises one or more atoms selected form O and N, preferably one or two atoms selected from O and N, more preferably exactly one atom selected from O and N, wherein each bridging cycloalkyl or bridging heterocycloalkyl is optionally substituted by one or more —C₁-C₃ alkyl or —C₁-C₃ alkoxy, more preferably —C₁-C₂ alkyl or —C₁-C₂ alkoxy; or
(iii) X³ and X⁶ are both chemical bonds; R^{1A}, R², R^{2A}, R^{4A}, R⁵, and R^{5A} are —H; one of R¹ and R⁴ combine with R⁵¹ to form a bridging 5-7 membered heterocycloalkyl ring wherein the heterocycloalkyl ring comprises one or more atoms selected form O and N, preferably one or two atoms selected from O and N, wherein the bridging heterocycloalkyl is optionally substituted by one or more —C₁-C₃ alkyl or —C₁-C₃ alkoxy, more preferably —C₁-C₂ alkyl or —C₁-C₂ alkoxy; wherein
when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl wherein the second bridging heterocycloalkyl comprises one or more atoms selected form O and N, preferably one or two atoms selected from O and N and is optionally substituted by one or more —C₁-C₃ alkyl or —C₁-C₃ alkoxy, more preferably —C₁-C₂ alkyl or —C₁-C₂ alkoxy; or
(iv) exactly one of X³ and X⁶ is a chemical bond, optionally wherein any of R¹, R², R⁴, R⁵ and the remaining R³ or R⁶ can combine to form a bridging C₄-C₈ cycloalkyl or bridging 5 to 8-membered heterocycloalkyl.

In some embodiments, X¹ is CR¹R^{1A}; X² is CR²R^{2A}; X⁴ is CR⁴R^{4A}; X⁵ is CR⁵R^{5A};
(i) X³ and X⁶ are both chemical bonds; R^{1A}, R², R^{2A}, R^{4A}, R⁵, and R^{5A} are —H; R¹ and R⁴ combine to form a first bridging C₄-C₆ cycloalkyl ring or a first bridging 5-7 membered heterocycloalkyl ring, wherein the heterocycloalkyl ring comprises one or more atoms selected form O and N, preferably one or two atoms selected from O and N, more preferably exactly one atom selected from O and N, wherein each bridging cycloalkyl or bridging heterocycloalkyl is optionally substituted by one or more —C₁-C₃ alkyl or —C₁-C₃ alkoxy, more preferably —C₁-C₂ alkyl or —C₁-C₂ alkoxy; or
(ii) X³ and X⁶ are both chemical bonds; R^{1A}, R¹, R^{2A}, R^{4A}, R⁴, and R^{5A} are —H, R² and R⁵ combine to form a first bridging C₅-C₇ cycloalkyl ring or a first bridging 5-7 membered heterocycloalkyl ring, wherein the heterocycloalkyl ring comprises one or more atoms selected form O and N, preferably one or two atoms selected from O and N, more preferably exactly one atom selected from O and N, wherein each bridging cycloalkyl or bridging heterocycloalkyl is optionally substituted by one or more —C₁-C₃ alkyl or —C₁-C₃ alkoxy, more preferably —C₁-C₂ alkyl or —C₁-C₂ alkoxy; or
(iii) X³ and X⁶ are both chemical bonds; R^{1A}, R², R^{2A}, R^{4A}, R⁵, and R^{5A} are —H; one of R¹ and R⁴ combine with R⁵¹ to form a bridging 5-7 membered heterocycloalkyl ring wherein the heterocycloalkyl ring comprises one or more atoms selected form O and N, preferably one or two atoms selected from O and N, wherein the bridging heterocycloalkyl is optionally substituted by one or more —C₁-C₃ alkyl or —C₁-C₃ alkoxy, more preferably —C₁-C₂ alkyl or —C₁-C₂ alkoxy; wherein
when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl wherein the second bridging heterocycloalkyl comprises one or more atoms selected form O and N, preferably one or two atoms selected from O and N and is optionally substituted by one or more —C₁-C₃ alkyl or —C₁-C₃ alkoxy, more preferably —C₁-C₂ alkyl or —C₁-C₂ alkoxy; or
(iv) exactly one of X³ and X⁶ is a chemical bond.

In some embodiments, X¹ is CR¹R^{1A}; X² is CR²R^{2A}; X⁴ is CR⁴R^{4A}; X⁵ is CR⁵R^{5A};
(i) X³ and X⁶ are both chemical bonds; R^{1A}, R², R^{2A}, R^{4A}, R⁵, and R^{5A} are —H; R¹ and R⁴ combine to form a first bridging cyclobutyl, cyclopentyl, cyclohexyl, or piperidinyl ring, wherein each cyclobutyl, cyclopentyl, cyclohexyl, or piperidinyl ring is optionally substituted with one or more —C₁-C₃ alkyl or —C₁-C₃ alkoxy, more preferably —C₁-C₂ alkyl or —C₁-C₂ alkoxy; or
(ii) X³ and X⁶ are both chemical bonds; R^{1A}, R¹, R^{2A}, R^{4A}, R⁴, and R^{5A} are —H; R² and R⁵ combine to form a first bridging cyclohexyl or cycloheptyl ring, wherein each cyclohexyl or cycloheptyl is optionally substituted with one or more —C₁-C₃ alkyl or —C₁-C₃ alkoxy, more preferably —C₁-C₂ alkyl or —C₁-C₂ alkoxy; or
(iii) X³ and X⁶ are both chemical bonds; R^{1A}, R², R^{2A}, R^{4A}, R⁵, and R^{5A} are —H; one of R¹ and R⁴ combine with R⁵¹ to form a bridging pyrrolidinyl, piperidinyl, or oxazinanyl preferably a bridging pyrrolidinyl, piperidinyl, or 1,4 oxazinanyl, wherein each pyrrolidinyl, piperidinyl, or oxazinanyl is optionally substituted with one or more —C₁-C₃ alkyl or —C₁-C₃ alkoxy, more preferably —C₁-C₂ alkyl or —C₁-C₂ alkoxy; wherein when R¹ and R⁴ or R² and R⁵ combine to form a first bridging cyclohexyl or piperidinyl, the first bridging cyclohexyl or piperidinyl can independently combine with R⁵¹ to form a second bridging piperidinyl or 1,3-diazinanyl, wherein the second bridging piperidinyl or 1,3-diazinanyl is optionally substituted by one or more —C₁-C₃ alkyl or —C₁-C₃ alkoxy, more preferably —C₁-C₂ alkyl or —C₁-C₂ alkoxy; or
(iv) exactly one of X³ and X⁶ is a chemical bond, optionally wherein any of R¹, R², R⁴, R⁵ and the remaining R³ or R⁶ can combine to form a bridging C₄-C₈ cycloalkyl or bridging 5 to 8-membered heterocycloalkyl.

In some embodiments, X¹ is CR¹R^{1A}; X² is CR²R^{2A}; X⁴ is CR⁴R^{4A}; X⁵ is CR⁵R^{5A};
(i) X³ and X⁶ are both chemical bonds; R^{1A}, R², R^{2A}, R^{4A}, R⁵, and R^{5A} are —H; R¹ and R⁴ combine to form a first bridging cyclobutyl, cyclopentyl, cyclohexyl, or piperidinyl ring, wherein each cyclobutyl, cyclopentyl, cyclohexyl, or piperidinyl ring is optionally substituted with one or more —C₁-C₃ alkyl or —C₁-C₃ alkoxy, more preferably —C₁-C₂ alkyl or —C₁-C₂ alkoxy; or
(ii) X³ and X⁶ are both chemical bonds; R^{1A}, R¹, R^{2A}, R^{4A}, R⁴, and R^{5A} are —H; R² and R⁵ combine to form a first bridging cyclohexyl or cycloheptyl ring, wherein each cyclohexyl or cycloheptyl is optionally substituted with one or more —C₁-C₃ alkyl or —C₁-C₃ alkoxy, more preferably —C₁-C₂ alkyl or —C₁-C₂ alkoxy; or
(iii) X³ and X⁶ are both chemical bonds; R^{1A}, R², R^{2A}, R^{4A}, R⁵, and R^{5A} are —H; one of R¹ and R⁴ combine with R⁵¹ to form a bridging pyrrolidinyl, piperidinyl, or oxazinanyl preferably a bridging pyrrolidinyl, piperidinyl, or 1,4 oxazinanyl, wherein each pyrrolidinyl, piperidinyl, or oxazinanyl is optionally substituted with one or more —C₁-C₃ alkyl or —C₁-C₃ alkoxy, more preferably —C₁-C₂ alkyl or —C₁-C₂ alkoxy; wherein when R¹ and R⁴ or R² and R⁵ combine to form a first bridging cyclohexyl or piperidinyl, the first bridging cyclohexyl or piperidinyl can independently combine with R⁵¹ to form a second bridging piperidinyl or 1,3-diazinanyl, wherein the second bridging piperidinyl or 1,3-diazinanyl is optionally substituted by one or more —C₁-C₃ alkyl or —C₁-C₃ alkoxy, more preferably —C₁-C₂ alkyl or —C₁-C₂ alkoxy; or
(iv) exactly one of X³ and X⁶ is a chemical bond.

In some embodiments, X¹ is CR¹R^{1A}; X² is CR²R^{2A}; X⁴ is CR⁴R^{4A}; X⁵ is CR⁵R^{5A};
(i) X³ and X⁶ are both chemical bonds; R^{1A}, R², R^{2A}, R^{4A}, R⁵, and R^{5A} are —H; R¹ and R⁴ combine to form a first bridging cyclobutyl, cyclopentyl, cyclohexyl, or piperidinyl ring, wherein each cyclobutyl, cyclopentyl, cyclohexyl, or piperidinyl ring is optionally substituted with one or more —C₁-C₃ alkyl, preferably one or two —C₁-C₂ alkyl; or
(ii) X³ and X⁶ are both chemical bonds; R^{1A}, R¹, R^{2A}, R^{4A}, R⁴, and R^{5A} are —H; R² and R⁵ combine to form a first bridging cyclohexyl or cycloheptyl ring, wherein each cyclohexyl or cycloheptyl is optionally substituted with one or more —C₁-C₃ alkyl, preferably one or two —C₁-C₂ alkyl; or
(iii) X³ and X⁶ are both chemical bonds; R^{1A}, R², R^{2A}, R^{4A}, R⁵, and R^{5A} are —H; one of R¹ and R⁴ combine with R⁵¹ to form a bridging pyrrolidinyl, piperidinyl, or oxazinanyl, preferably a bridging pyrrolidinyl, piperidinyl, or 1,4 oxazinanyl, wherein each pyrrolidinyl, piperidinyl, or oxazinanyl is optionally substituted with one or more —C₁-C₃ alkyl, preferably one or two —C₁-C₂ alkyl; wherein when R¹ and R⁴ or R² and R⁵ combine to form a first bridging cyclohexyl or piperidinyl, the first bridging cyclohexyl or piperidinyl can independently combine with R⁵¹ to form a second bridging piperidinyl or 1,3-diazinanyl, wherein the second bridging piperidinyl or 1,3-diazinanyl is optionally substituted by one or more —C₁-C₃ alkyl, preferably one or two —C₁-C₂ alkyl; or
(iv) exactly one of X³ and X⁶ is a chemical bond, optionally wherein any of R¹, R², R⁴, R⁵ and the remaining R³ or R⁶ can combine to form a bridging C₄-C₈ cycloalkyl or bridging 5 to 8-membered heterocycloalkyl.

In some embodiments, X¹ is CR¹R^{1A}; X² is CR²R^{2A}; X⁴ is CR⁴R^{4A}; X⁵ is CR⁵R^{5A};
(i) X³ and X⁶ are both chemical bonds; R^{1A}, R², R^{2A}, R^{4A}, R⁵, and R^{5A} are —H; R¹ and R⁴ combine to form a first bridging cyclobutyl, cyclopentyl, cyclohexyl, or piperidinyl ring, wherein each cyclobutyl, cyclopentyl, cyclohexyl, or piperidinyl ring is optionally substituted with one or more —C₁-C₃ alkyl, preferably one or two —C₁-C₂ alkyl; or
(ii) X³ and X⁶ are both chemical bonds; R^{1A}, R¹, R^{2A}, R^{4A}, R⁴, and R^{5A} are —H; R² and R⁵ combine to form a first bridging cyclohexyl or cycloheptyl ring, wherein each cyclohexyl or cycloheptyl is optionally substituted with one or more —C₁-C₃ alkyl, preferably one or two —C₁-C₂ alkyl; or
(iii) X³ and X⁶ are both chemical bonds; R^{1A}, R², R^{2A}, R^{4A}, R⁵, and R^{5A} are —H; one of R¹ and R⁴ combine with R⁵¹ to form a bridging pyrrolidinyl, piperidinyl, or oxazinanyl, preferably a bridging pyrrolidinyl, piperidinyl, or 1,4 oxazinanyl, wherein each pyrrolidinyl, piperidinyl, or oxazinanyl is optionally substituted with one or more —C₁-C₃ alkyl, preferably one or two —C₁-C₂ alkyl; wherein when R¹ and R⁴ or R² and R⁵ combine to form a first bridging cyclohexyl or piperidinyl, the first bridging cyclohexyl or piperidinyl can independently combine with R⁵¹ to form a second bridging piperidinyl or 1,3-diazinanyl, wherein the second bridging piperidinyl or 1,3-diazinanyl is optionally substituted by one or more —C₁-C₃ alkyl, preferably one or two —C₁-C₂ alkyl; or
(iv) exactly one of X³ and X⁶ is a chemical bond.

In some embodiments, R⁵¹ is selected from —H, —C₁-C₄ alkyl, and —C₅-C₆ cycloalkyl.

In some embodiments, R⁵¹ is selected from —H, methyl, ethyl, propyl, and tert-butyl.

In some embodiments, exactly one of X³ and X⁶ is a chemical bond.

In some embodiments, exactly one of X², X³, X⁵, and X⁶ is a chemical bond.

In some embodiments, exactly one of X³ and X⁶ is a chemical bond and X¹, X², X⁴, and X⁵ are each CR¹R^{1A}, CR²R^{2A}, CR⁴R^{4A}, CR⁵R^{5A}.

In some embodiments, X³ is a chemical bond, and R⁵¹ is —H, —C₁-C₆ alkyl, preferably —H or —C₁-C₃ alkyl, more preferably —H or —C₁-C₂ alkyl.

In some embodiments, X³ is a chemical bond; X¹, X², X⁴, X⁵, and X⁶ are each CR¹R^{1A}CR²R^{2A}, CR⁴R^{4A}, CR⁵R^{5A} and CR⁶R^{6A}, wherein R¹, R^{1A}, R², R^{2A}, R⁴, R^{4A}, R⁵, R^{5A}, R⁶ and R^{6A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X³ is a chemical bond; X¹, X², X⁴, X⁵, and X⁶ are each CR¹R^{1A}CR²R^{2A}, CR⁴R^{4A}, CR⁵R^{5A} and CR⁶R^{6A}, wherein R² and R⁶ combine to form a bridging —C₅-C₈ cycloalkyl, preferably a bridging —C₇-C₈ cycloalkyl; and wherein R¹, R^{1A}, R^{2A}, R⁴, R^{4A}, R⁵, R^{5A} and R^{6A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X³ is a chemical bond; X¹, X², X⁴, X⁵, and X⁶ are each CR¹R^{1A}CR²R^{2A}, CR⁴R^{4A}, CR⁵R^{5A} and CR⁶R^{6A}, wherein R² and R⁵ combine to form a bridging —C₅-C₈ cycloalkyl, preferably a bridging —C₆-C₇ cycloalkyl; and wherein R¹, R^{1A}, R^{2A}, R⁴, R^{4A}, R^{5A}, R⁶ and R^{6A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X³ is a chemical bond; X¹, X², X⁴, X⁵, and X⁶ are each CR¹R^{1A}CR²R^{2A}, CR⁴R^{4A}, CR⁵R^{5A} and CR⁶R^{6A}, wherein R² and R⁴ combine to form a bridging —C₅-C₈ cycloalkyl, preferably a bridging —C₅-C₆ cycloalkyl; and wherein R¹, R^{1A}, R^{2A}, R^{4A}, R⁵, R^{5A}, R⁶ and R^{6A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X³ is a chemical bond; X¹, X², X⁴, X⁵, and X⁶ are each CR¹R^{1A}CR²R^{2A}, CR⁴R^{4A}, CR⁵R^{5A} and CR⁶R^{6A}, wherein R¹ and R⁶ combine to form a bridging —C₅-C₈ cycloalkyl, preferably a bridging —C₆-C₇ cycloalkyl; and wherein R^{1A}, R², R^{2A}, R⁴, R^{4A}, R⁵, R^{5A} and R^{6A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X³ is a chemical bond; X¹, X², X⁴, X⁵, and X⁶ are each CR¹R^{1A}CR²R^{2A}, CR⁴R^{4A}, CR⁵R^{5A} and CR⁶R^{6A}, wherein R¹ and R⁵ combine to form a bridging —C₅-C₈ cycloalkyl, preferably a bridging —C₅-C₆ cycloalkyl; and wherein R^{1A}, R², R^{2A}, R⁴, R^{4A}, R^{5A}, R⁶ and R^{6A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X³ is a chemical bond; X¹, X², X⁴, X⁵, and X⁶ are each CR¹R^{1A}CR²R^{2A}, CR⁴R^{4A}, CR⁵R^{5A} and CR⁶R^{6A}, wherein R¹ and R⁴ combine to form a bridging —C₅-C₈ cycloalkyl, preferably a bridging —C₅ cycloalkyl; and wherein R^{1A}, R², R^{2A}, R^{4A}, R⁵, R^{5A}, R⁶ and R^{6A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X⁶ is a chemical bond, and R⁵¹ is —H, —C₁-C₆ alkyl, preferably —H or —C₁-C₃ alkyl, more preferably —H or —C₁-C₂ alkyl.

In some embodiments, X⁶ is a chemical bond; X¹, X², X³, X⁴, and X⁵ are each CR¹R^{1A}CR²R^{2A}, CR³R^{3A}, CR⁴R^{4A} and CR⁵R^{5A}, wherein R¹, R^{1A}, R², R^{2A}, R³, R^{3A}, R⁴, R^{4A}, R⁵ and R^{5A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X⁶ is a chemical bond; X¹, X², X³, X⁴, and X⁵ are each CR¹R^{1A}CR²R^{2A}, CR³R^{3A}, CR⁴R^{4A} and CR⁵R^{5A}, wherein R³ and R⁵ combine to form a bridging —C₅-C₈ cycloalkyl, preferably a bridging —C₇-C₈ cycloalkyl; and wherein R¹, R^{1A}, R², R^{2A}, R³, R⁴, R^{4A} and R^{5A} are each —H or —C₁-C₆ alkyl, preferably —H or —C₁-C₂ alkyl, more preferably —H; and
R⁵¹ is —H or —C₁-C₆ alkyl, preferably —H or —C₁-C₃ alkyl, more preferably —H or —C₁-C₂ alkyl.

In some embodiments, X⁶ is a chemical bond; X¹, X², X³, X⁴, and X⁵ are each CR¹R^{1A}CR²R^{2A}, CR³R^{3A}, CR⁴R^{4A} and CR⁵R^{5A}, wherein R² and R⁵ combine to form a bridging —C₅-C₈ cycloalkyl, preferably a bridging —C₆-C₇ cycloalkyl; and wherein R¹, R^{1A}, R^{2A}, R³, R^{3A}, R⁴, R^{4A} and R^{5A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X⁶ is a chemical bond; X¹, X², X³, X⁴, and X⁵ are each CR¹R^{1A}CR²R^{2A}, CR³R^{3A}, CR⁴R^{4A} and CR⁵R^{5A}, wherein R¹ and R⁵ combine to form a bridging —C₅-C₈ cycloalkyl, preferably a bridging —C₅-C₆ cycloalkyl; and wherein R^{1A}, R², R^{2A}, R³, R^{3A}, R⁴, R^{4A} and R^{5A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X⁶ is a chemical bond; X¹, X², X³, X⁴, and X⁵ are each CR¹R^{1A} CR²R^{2A}, CR³R^{3A}, CR⁴R^{4A} and CR⁵R^{5A}, wherein R³ and R⁴ combine to form a bridging —C₅-C₈ cycloalkyl, preferably a bridging —C₆-C₇ cycloalkyl; and wherein R¹, R^{1A}, R², R^{2A}, R^{3A}, R^{4A}, R⁵, and R^{5A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X⁶ is a chemical bond; X¹, X², X³, X⁴, and X⁵ are each CR¹R^{1A}CR²R^{2A}, CR³R^{3A}, CR⁴R^{4A} and CR⁵R^{5A}, wherein R² and R⁴ combine to form a bridging —C₅-C₈ cycloalkyl, preferably a bridging —C₅-C₆ cycloalkyl; and wherein R¹, R^{1A}, R^{2A}, R³, R^{3A}, R^{4A}, R⁵, and R^{5A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X⁶ is a chemical bond; X¹, X², X³, X⁴, and X⁵ are each CR¹R^{1A}CR²R^{2A}, CR³R^{3A}, CR⁴R^{4A} and CR⁵R^{5A}, wherein R¹ and R⁴ combine to form a bridging —C₅-C₈ cycloalkyl, preferably a bridging —C₅ cycloalkyl; and wherein R^{1A}, R², R^{2A}, R³, R^{3A}, R^{4A}, R⁵, and R^{5A} are each —H or —C₁-C₆ alkyl, preferably —H or —C₁-C₂ alkyl, more preferably —H; and R⁵¹ is —H or —C₁-C₆ alkyl, preferably —H or —C₁-C₃ alkyl, more preferably —H or —C₁-C₂ alkyl.

In some embodiments, X³ and X⁶ are both chemical bonds.

In some embodiments, X³ and X⁶ are both chemical bonds and X¹, X², X⁴, and X⁵ are each CR¹R^{1A}, CR²R^{2A}, CR⁴R^{4A}, and CR⁵R^{5A}.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R¹ and R⁴ combine to form a bridging C₄-C₈ cycloalkyl, preferably a C₄-C₆ cycloalkyl, more preferably a C₅-C₆ cycloalkyl, and wherein R^{1A}, R², R^{2A}, R^{4A}, R⁵, and R^{5A} are each —H or —C₁-C₆ alkyl, preferably —H or —C₁-C₂ alkyl, more preferably —H; and R⁵¹ is —H, —C₁-C₆ alkyl, or —C₃-C₈ cycloalkyl, preferably —H, —C₁-C₄ alkyl or C₅-C₆ cycloalkyl, more preferably —H, —C₁-C₂ alkyl or —C₅-C₆ cycloalkyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R¹ and R⁴ combine to form a bridging C₄-C₈ cycloalkyl, preferably a C₄-C₆ cycloalkyl, more preferably a C₅-C₆ cycloalkyl, and wherein R^{1A}, R², R^{2A}, R^{4A}, R⁵, and R^{5A} are each —H or —C₁-C₆ alkyl, preferably —H or —C₁-C₂ alkyl, more preferably —H; and R⁵¹ is —H or —C₁-C₆ alkyl, preferably —H or —C₁-C₄ alkyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R¹ and R⁴ combine to form a bridging C₅-C₈ heterocycloalkyl, preferably a bridging C₆ heterocycloalkyl, wherein said bridging heterocycloalkyl is optionally substituted with one or more —C₁-C₆ alkyl, preferably —C₁-C₃ alkyl; R^{1A}, R², R^{2A}, R^{4A}, R⁵, and R^{5A} are each —H or —C₁-C₆ alkyl, preferably —H or —C₁-C₂ alkyl, more preferably —H; and R⁵¹ is —H, or —C₁-C₆ alkyl, preferably —H, or —C₁-C₄ alkyl, more preferably —H, or —C₁-C₃ alkyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R¹ and R⁴ combine to form a bridging C₅-C₈ heterocycloalkyl, preferably a bridging C₆ heterocycloalkyl, wherein said bridging heterocycloalkyl comprises one or more heteroatoms selected from N and O, preferably one or two heteroatoms selected from N and O, preferably one heteroatom selected from N and O and is optionally substituted with one or more —C₁-C₆ alkyl, preferably —C₁-C₃ alkyl; R^{1A}, R², R^{2A}, R^{4A}, R⁵, and R^{5A} are each —H or —C₁-C₆ alkyl, preferably —H or —C₁-C₂ alkyl, more preferably —H; and R⁵¹ is —H, or —C₁-C₆ alkyl, preferably —H, or —C₁-C₄ alkyl, more preferably —H, or —C₁-C₃ alkyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R¹ and R⁴ combine to form a bridging piperidine, wherein said piperidine is optionally substituted at N with —C₁-C₆ alkyl, preferably —C₁-C₃ alkyl; R^{1A}, R², R^{2A}, R^{4A}, R⁵, and R^{5A} are each —H or —C₁-C₆ alkyl, preferably —H or —C₁-C₂ alkyl, more preferably —H; and R⁵¹ is —H, or —C₁-C₆ alkyl, preferably —H, or —C₁-C₄ alkyl, more preferably —H, or —C₁-C₃ alkyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R¹ and R⁴ combine to form a bridging C₄-C₈ cycloalkyl or bridging C₄-C₈ heterocycloalkyl, preferably a C₄-C₆ cycloalkyl or C₄-C₆ heterocycloalkyl, and wherein R^{1A}, R², R^{2A}, R^{4A}, R⁵, and R^{5A} are each —H or —C₁-C₆ alkyl, preferably —H or —C₁-C₂ alkyl, more preferably —H; and R⁵¹ is connected to the bridging C₄-C₆ cycloalkyl or C₄-C₆ heterocycloalkyl formed by R¹ and R⁴.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R¹ and R⁴ combine to form a bridging cyclohexane or cyclopentane, and wherein R^{1A}, R², R^{2A}, R^{4A}, R⁵, and R^{5A} are each —H or —C₁-C₆ alkyl, preferably —H or —C₁-C₂ alkyl, more preferably —H; and R⁵¹ is connected to the bridging cyclohexane or cyclopentane formed by R¹ and R⁴ by a —C₁-C₂ alkylene, preferably by a —C₁ alkylene.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R¹ and R⁴ combine to form a bridging piperidine or pyrrolidine, and wherein R^{1A}, R², R^{2A}, R^{4A}, R⁵, and R^{5A} are each —H or —C₁-C₆ alkyl, preferably —H or —C₁-C₂ alkyl, more preferably —H; and R⁵¹ is connected to the bridging piperidine or pyrrolidine formed by R¹ and R⁴ by a —C₁-C₂ alkylene, preferably by a —C₁ alkylene.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R² and R⁵ combine to form a bridging C₄-C₈ cycloalkyl, preferably a C₆-C₇ cycloalkyl; R¹, R^{1A}, R^{2A}, R⁴, R^{4A}, and R^{5A} are each —H or —C₁-C₆ alkyl, preferably —H or —C₁-C₂ alkyl, more preferably —H; and R⁵¹ is —H, —C₁-C₆ alkyl, or —C₃-C₈ cycloalkyl, preferably —H, —C₁-C₄ alkyl or C₅-C₆ cycloalkyl, more preferably —H, —C₁-C₃ alkyl or —C₅-C₆ cycloalkyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R² and R⁵ combine to form a bridging C₇ cycloalkyl; R¹, R^{1A}, R^{2A}, R⁴, R^{4A}, and R^{5A} are each —H or —C₁-C₆ alkyl, preferably —H or —C₁-C₂ alkyl, more preferably —H; and R⁵¹ is —H.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R² and R⁵ combine to form a bridging C₇ cycloalkyl; R¹, R^{1A}, R^{2A}, R⁴, R^{4A}, and R^{5A} are each —H or —C₁-C₆ alkyl, preferably —H or —C₁-C₂ alkyl, more preferably —H; and R⁵¹ is methyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R² and R⁵ combine to form a bridging C₇ cycloalkyl; R¹, R^{1A}, R^{2A}, R⁴, R^{4A}, and R^{5A} are each —H or —C₁-C₆ alkyl, preferably —H or —C₁-C₂ alkyl, more preferably —H; and R⁵¹ is isopropyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R² and R⁵ combine to form a bridging C₇ cycloalkyl; R, R^{1A}, R^{2A}, R⁴, R^{4A}, and R^{5A} are each —H or —C₁-C₆ alkyl, preferably —H or —C₁-C₂ alkyl, more preferably —H; and R⁵¹ is propyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R² and R⁵ combine to form a bridging C₇ cycloalkyl; R¹, R^{1A}, R^{2A}, R⁴, R^{4A}, and R^{5A} are each —H or —C₁-C₆ alkyl, preferably —H or —C₁-C₂ alkyl, more preferably —H; and R⁵¹ is cyclopentyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein R² and R⁵ combine to form a bridging C₆ cycloalkyl; R¹, R^{1A}, R^{2A}, R⁴, R^{4A}, and R^{5A} are each —H or —C₁-C₆ alkyl, preferably —H or —C₁-C₂ alkyl, more preferably —H; and R⁵¹ is methyl.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein one of R¹ and R⁴ combine to form a bridging C₄-C₈ cycloalkyl or 4-8 membered heterocycloalkyl with R⁵¹, preferably a C₅-C₆ cycloalkyl or 5-6 membered heterocycloalkyl, and wherein R^{1A}, R², R^{2A}, R^{4A}, R⁵, and R^{5A} are each —H or —C₁-C₆ alkyl, preferably —H or —C₁-C₂ alkyl, more preferably —H.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein one of R¹ and R⁴ combine to form a bridging C₄-C₈ cycloalkyl with R⁵¹, preferably a C₅-C₆ cycloalkyl, and wherein R^{1A}, R², R^{2A}, R^{4A}, R⁵, and R^{5A} are each —H or —C₁-C₆ alkyl, preferably —H or —C₁-C₂ alkyl, more preferably —H.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein one of R¹ and R⁴ combine to form a bridging 4-8 membered heterocycloalkyl with R⁵¹, preferably a 5-6 membered heterocycloalkyl, and wherein R^{1A}, R², R^{2A}, R^{4A}, R⁵, and R^{5A} are each —H or —C₁-C₆ alkyl, preferably —H or —C₁-C₂ alkyl, more preferably —H.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein one of R¹ and R⁴ combine to form a bridging 4-8 membered heterocycloalkyl with R⁵¹, preferably a 5-6 membered heterocycloalkyl, wherein the bridging heterocycloalkyl comprises one or more heteroatoms selected from N and O, preferably one or two heteroatoms selected from N and O, preferably one heteroatom selected from N and O and wherein R^{1A}, R², R^{2A}, R^{4A}, R⁵, and R^{5A} are each —H or —C₁-C₆ alkyl, preferably —H or —C₁-C₂ alkyl, more preferably —H.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein one of R¹ and R⁴ combine to form a bridging 1,3 oxazine ring, and wherein R^{1A}, R², R^{2A}, R^{4A}, R⁵, and R^{5A} are each —H or —C₁-C₆ alkyl, preferably —H or —C₁-C₂ alkyl, more preferably —H.

In some embodiments, X³ and X⁶ are both chemical bonds; X¹, X², X⁴, and X⁵ are each CR¹R^{1A}, CR²R^{2A}, CR⁴R^{4A}, and CR⁵R^{5A}, wherein one of R¹ and R⁴ combine with R⁵¹ to form a bridging 1,4 oxazine ring, and wherein R^{1A}, R², R^{2A}, R^{4A}, R⁵, and R^{5A} are each —H or —C₁-C₆ alkyl, preferably —H or —C₁-C₂ alkyl, more preferably —H.

In some embodiments, the compound is of Formula IA, IB, IC, ID, or IE:

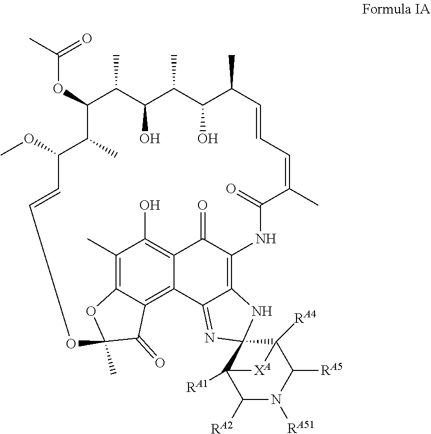

wherein:
R^{A1}, R^{A2}, R^{A4} and R^{A5} are each independently —H or —C₁-C₄ alkyl;
X^{A} is independently selected from —CH₂—, —CH₂CH₂—, —CH₂CH₂CH₂—, —CH₂OCH₂—, —CH₂NHCH₂—, or —CH₂N(C₁-C₄ alkyl)CH₂;
R^{A51} is independently —H or —C₁-C₄ alkyl; or
X^{A} and R^{A51}, together with the atoms to which they are attached, combine to form a 5 to 6-membered heterocycloalkyl;

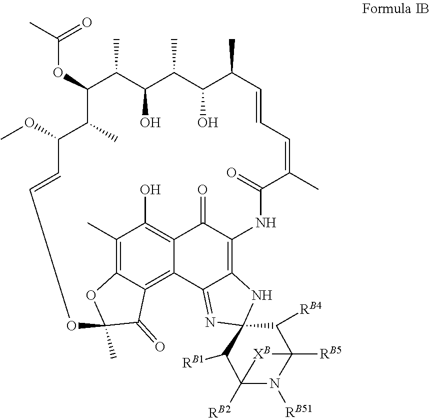

wherein:
R^{B1}, R^{B2}, R^{B4} and R^{B5} are each independently —H or —C₁-C₄ alkyl;
X^{B} is independently selected from —CH₂—, and —CH₂CH₂—;
R^{B51} is independently —H or —C₁-C₄ alkyl:

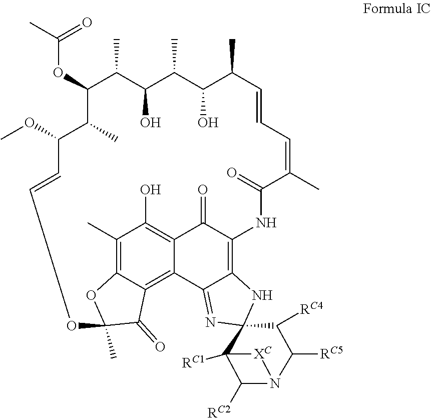

wherein:
R^{C1}, R^{C2}, R^{C4} and R^{C5} are each independently —H or —C₁-C₄ alkyl;
X° is independently selected from —CH₂CH₂—, —CH₂CH₂CH₂—, —OCH₂CH₂—, and —NHCH₂CH₂—;

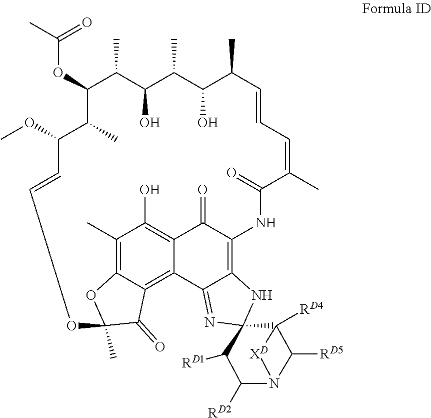

wherein:
R^{D1}, R^{D2}, R^{D4} and R^{D5} are each independently —H or —C₁-C₄ alkyl;
X^{D} is independently selected from —CH₂CH₂—, —CH₂CH₂CH₂—, —OCH₂CH₂—, and —NHCH₂CH₂—;

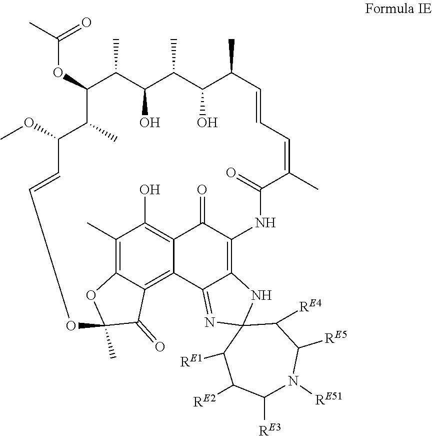

wherein:
R^{E1}, R^{E2}, and R^{E4} are each —H or C₁-C₄ alkyl; or
R^{E3} and R^{E5} are H, or combine to form a bridging cyclooctane; and;
R^{E51} is —H or —C₁-C₄ alkyl.

In some embodiments, the compound is of Formula IA. In some embodiments, the compound is of Formula IB. In some embodiments, the compound is of Formula IC. In some embodiments, the compound is of Formula ID. In some embodiments, the compound is of Formula IE.

In some embodiments, X¹, X², X³, X⁴, X⁵, X⁶ and NR¹ form any structure selected from:

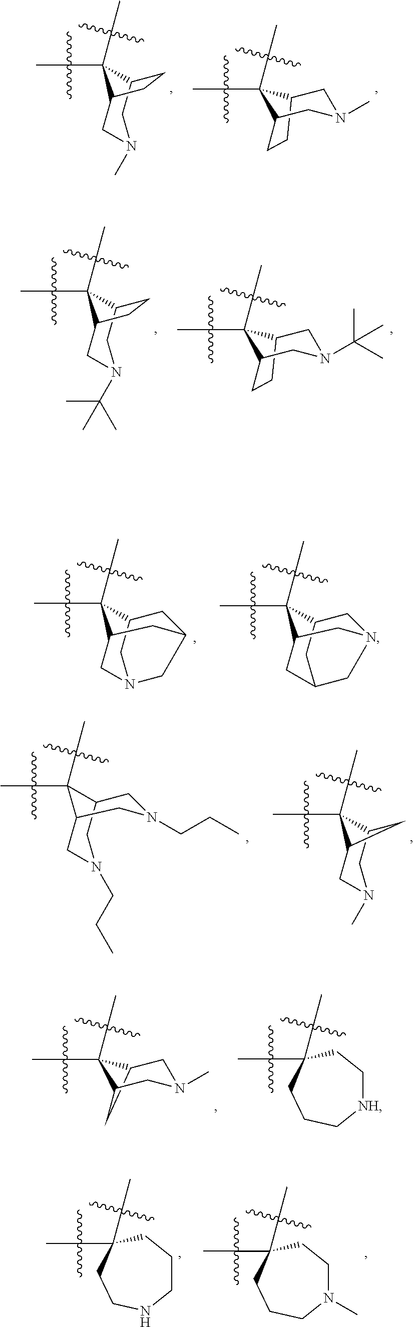

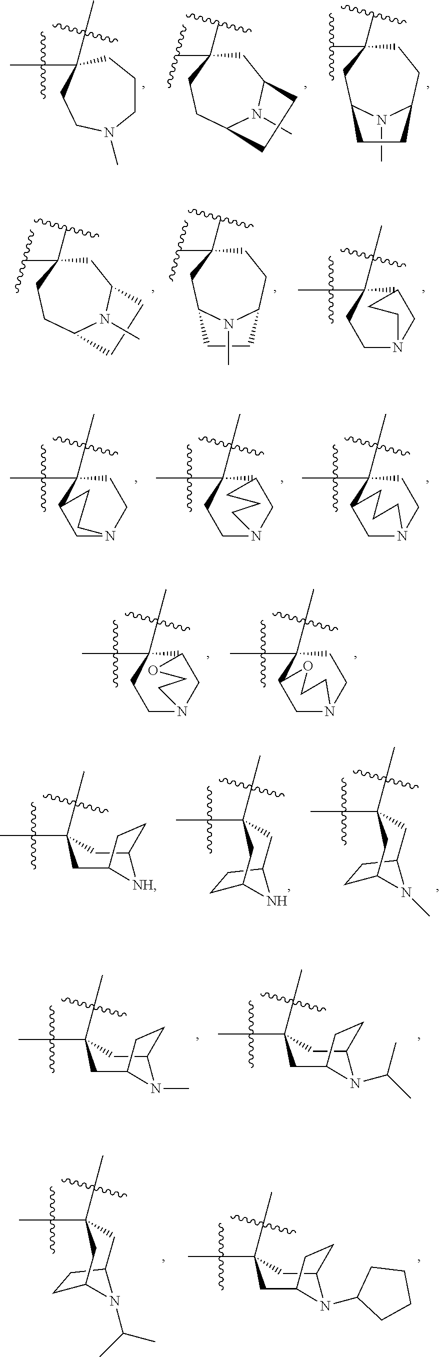

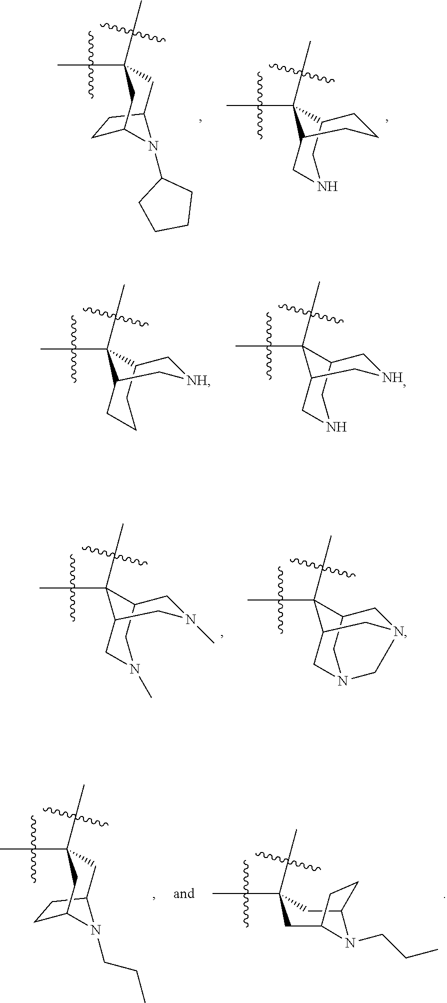

In some embodiments, the compound is selected from the group consisting of:

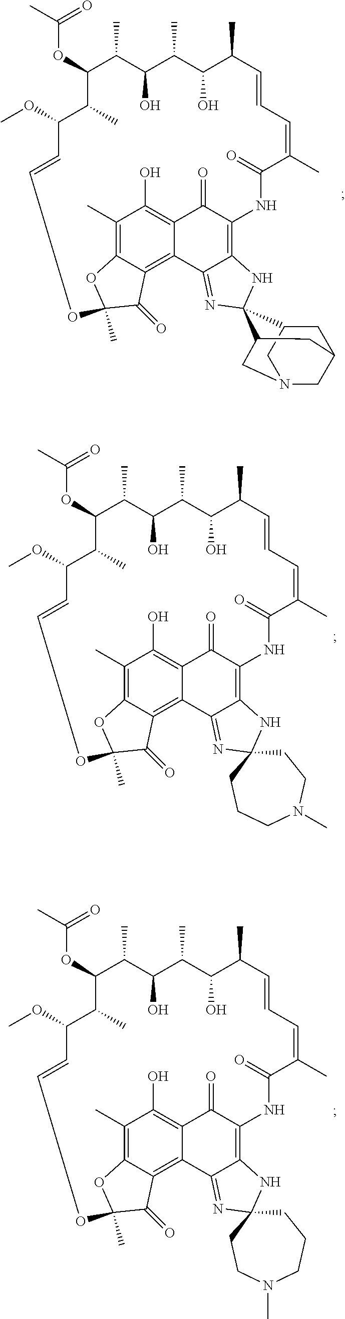

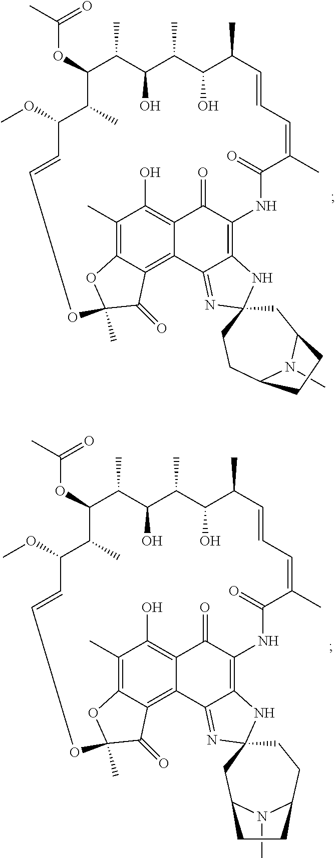

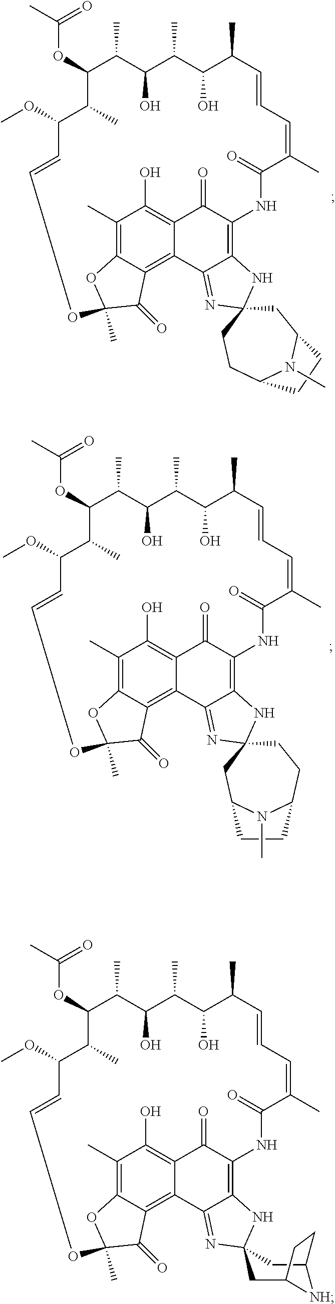

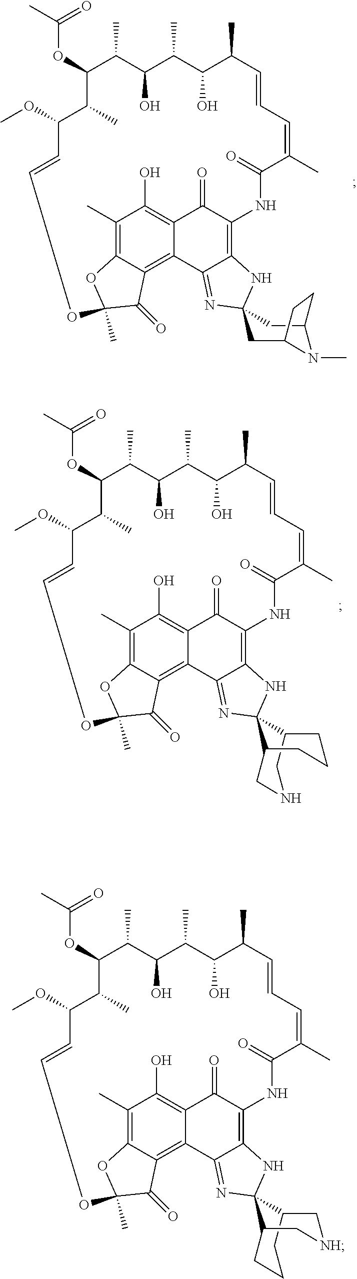

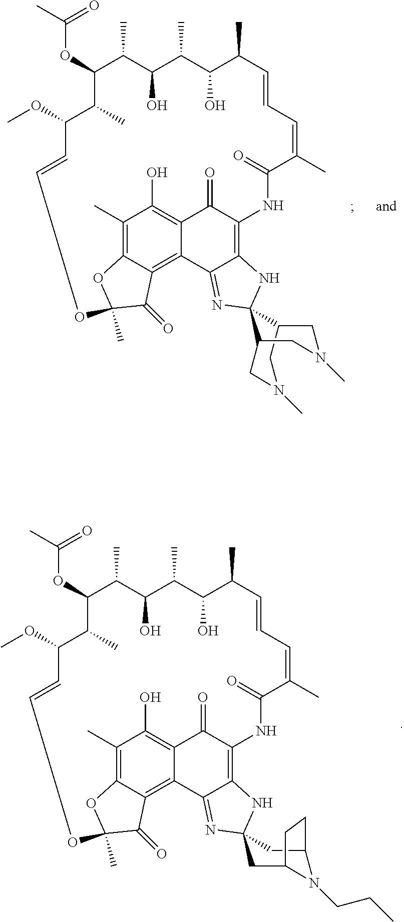

In some embodiments, the compound is selected from the group consisting of:

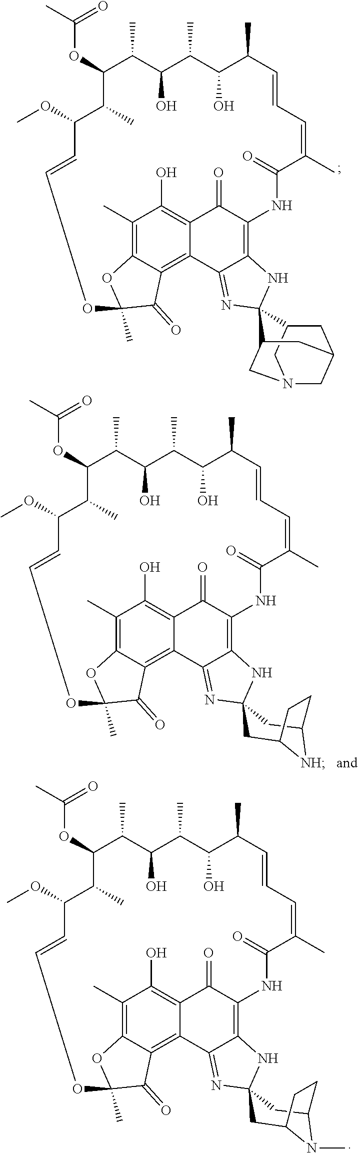

In some embodiments, the compound is:

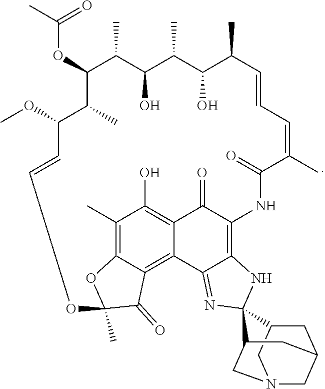

In some embodiments, the compound is:

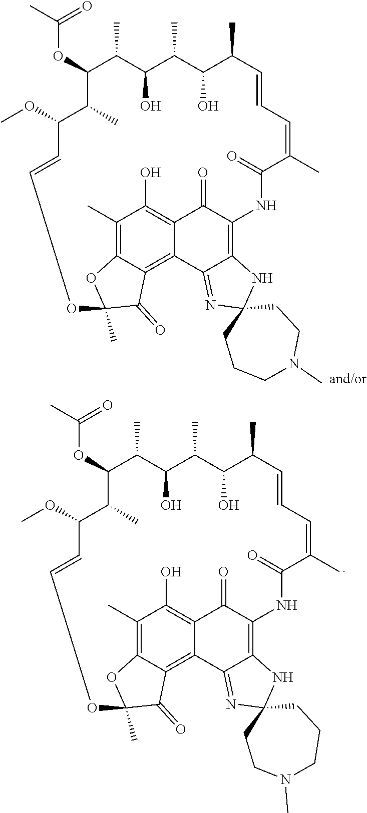

In some embodiments, the compound is:

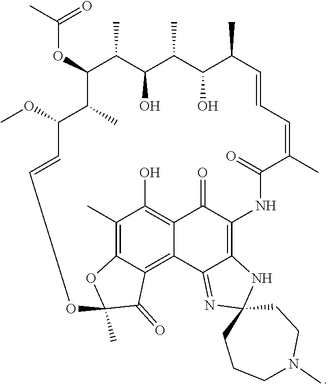

In some embodiments, the compound is:

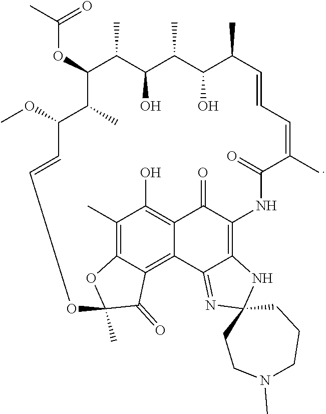

In some embodiments, the compound is:

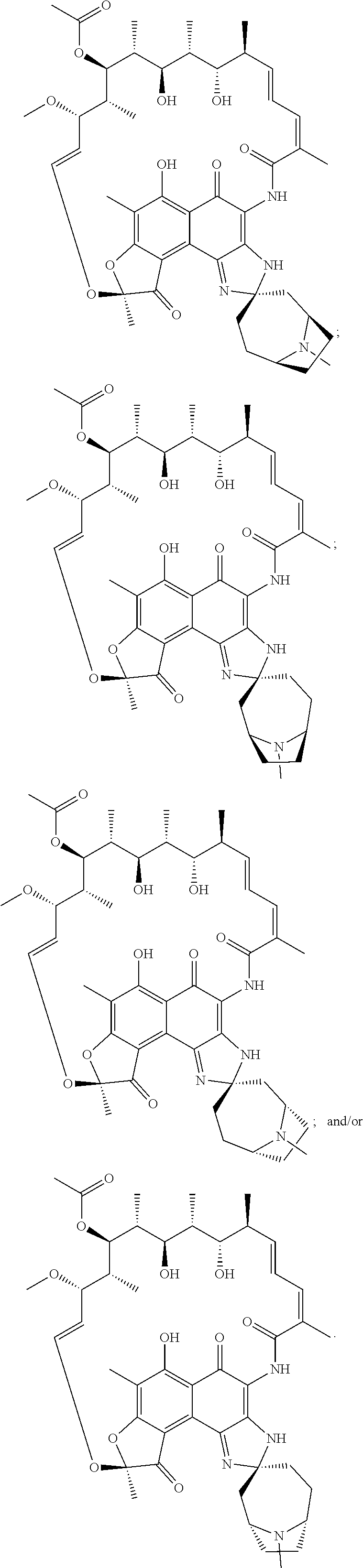

In some embodiments, the compound is:

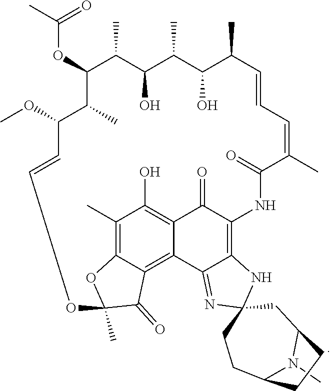 [SMILES: CO[C@H]1/C=C/O[C@@]2(C)Oc3c(C)c(O)c4c(c3C2=O)C2=N[C@]3(CC[C@@H]5CC[C@H](C3)N5C)NC2=C(NC(=O)/C(C)=C\C=C\[C@H](C)[C@H](O)[C@@H](C)[C@@H](O)[C@@H](C)[C@H](OC(C)=O)[C@@H]1C)C4=O]

In some embodiments, the compound is:

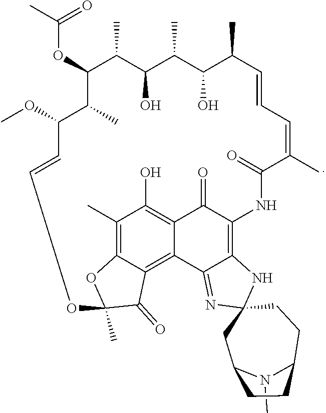

In some embodiments, the compound is:

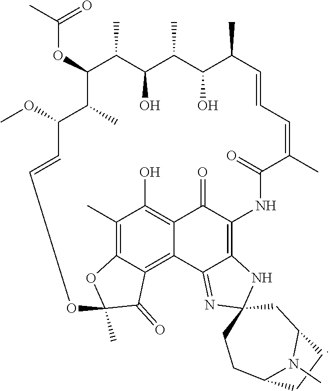 [SMILES: CO[C@H]1/C=C/O[C@@]2(C)Oc3c(C)c(O)c4c(c3C2=O)C2=N[C@]3(CC[C@H]5CC[C@@H](C3)N5C)NC2=C(NC(=O)/C(C)=C\C=C\[C@H](C)[C@H](O)[C@@H](C)[C@@H](O)[C@@H](C)[C@H](OC(C)=O)[C@@H]1C)C4=O]

In some embodiments, the compound is:

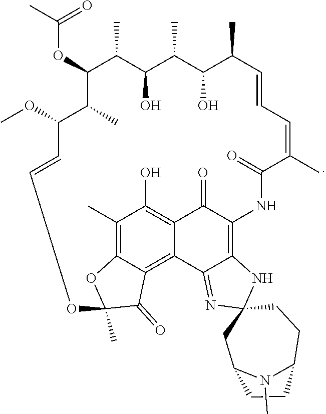

In some embodiments, the compound is:

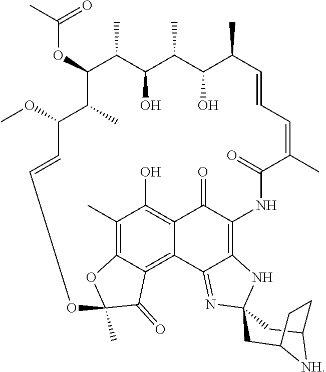

In some embodiments, the compound is:

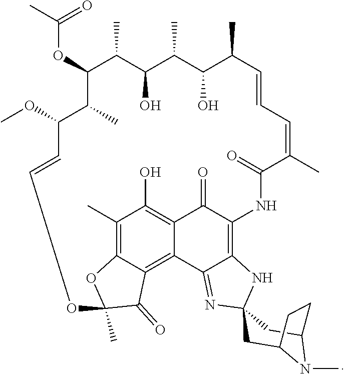

In some embodiments, the compound is:

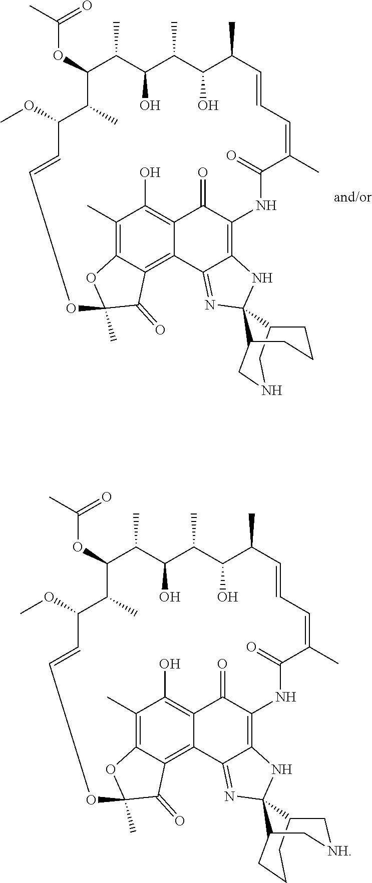

In some embodiments, the compound is:

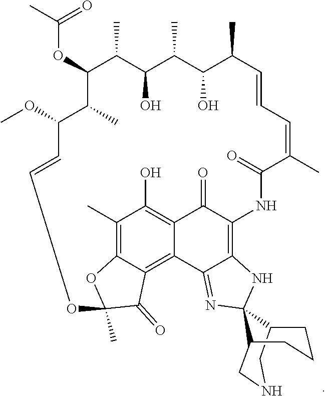

In some embodiments, the compound is:

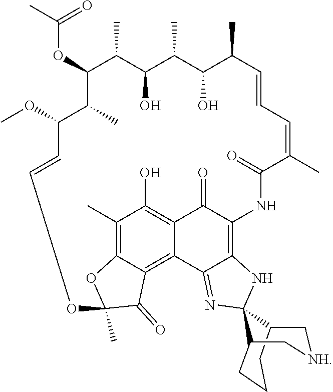

In some embodiments, the compound is:

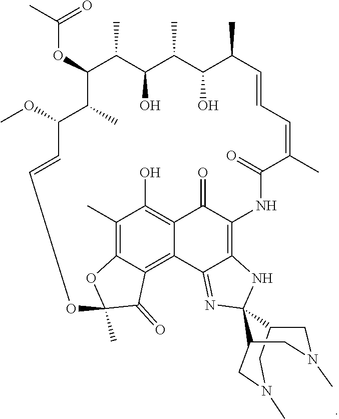

In some embodiments, the compound is:

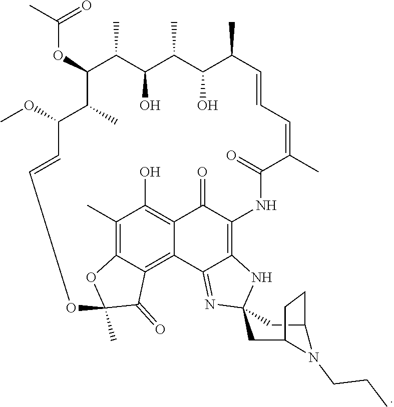

In some embodiments, the compound is Compound 1A.

In some embodiments, the compound is Compound 1B.

In some embodiments, the compound is Compound 2A.

In some embodiments, the compound is Compound 2B.

In some embodiments, the compound is Compound 3A.

In some embodiments, the compound is Compound 3B.

In some embodiments, the compound is Compound 4.

In some embodiments, the compound is Compound 5A.

In some embodiments, the compound is Compound 5B.

In some embodiments, the compound is Compound 6A.

In some embodiments, the compound is Compound 6B.

In some embodiments, the compound is Compound 7A.

In some embodiments, the compound is Compound 7B.

In some embodiments, the compound is Compound 8A.

In some embodiments, the compound is Compound 8B.

In some embodiments, the compound is Compound 8C.

In some embodiments, the compound is Compound 8D.

In some embodiments, the compound is Compound 9A.

In some embodiments, the compound is Compound 9B.

In some embodiments, the compound is Compound 10A.

In some embodiments, the compound is Compound 10B.

In some embodiments, the compound is Compound 11A.

In some embodiments, the compound is Compound 11B.

In some embodiments, the compound is Compound 12A.

In some embodiments, the compound is Compound 12B.

In some embodiments, the compound is Compound 13A.

In some embodiments, the compound is Compound 13B.

In some embodiments, the compound is Compound 14A.

In some embodiments, the compound is Compound 14B.

In some embodiments, the compound is Compound 15A.

In some embodiments, the compound is Compound 15B.

In some embodiments, the compound is Compound 16A.

In some embodiments, the compound is Compound 16B.

In some embodiments, the compound is Compound 17.

In some embodiments, the compound is Compound 18.

In some embodiments, the compound is Compound 19.

In some embodiments, the compound is Compound 20A.

In some embodiments, the compound is Compound 20B.

### Methods of Synthesizing the Disclosed Compounds

The compounds of the present disclosure can be prepared by methods known in the art of organic synthesis as set forth in part by the following synthetic schemes and examples below, together with synthetic methods known in the art of synthetic organic chemistry, or variations thereon as appreciated by those skilled in the art. The methods include but are not limited to those methods described below. Starting materials are either commercially available or made by known procedures reported in the literature or as illustrated.

In the schemes and examples described below, it is well understood that protecting groups for sensitive or reactive groups are employed where necessary in accordance with general principles of chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of the present disclosure.

Those skilled in the art will recognize if a stereocenter exists in the compounds of Formula I. Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley Interscience, 1994).

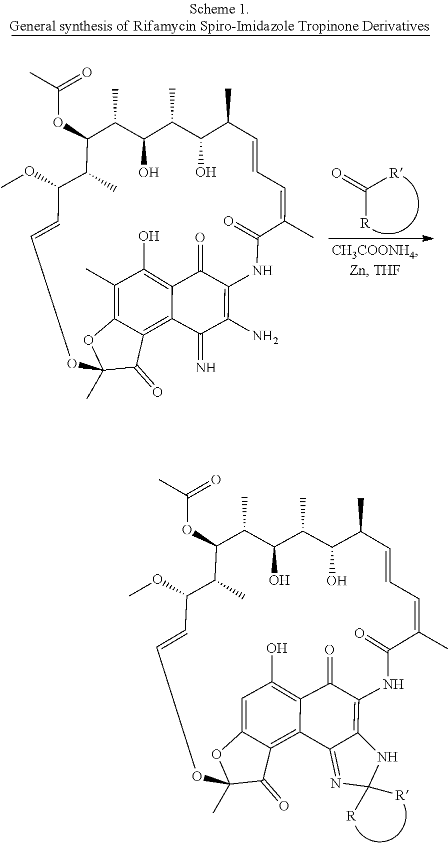

A general method of preparing compounds of Formula (I) is outlined in Scheme 1. To a suspension of ammonium acetate (2.5 eq) and zinc (2.5 eq) in THE was added 3-amino-4-deoxy-4-imino-rifamycin S (1 eq), ketone (2.5 eq) and acetic acid (20 eq). The reaction mixture was stirred at room temperature or elevated temperature and monitored by LC/MS. Upon completion, the mixture was filtered and evaporated. The desired compounds were either tested for antimicrobial activity without further purification or purified by flash chromatography and tested for antimicrobial activity.

### Methods of Using the Disclosed Compounds

The inventive compounds are rifamycin derivatives containing modified spiro-imidazole moieties at the 3,4-imino-amino positions and that exhibit broad spectrum antibacterial activity characteristic of the rifamycin class. Additionally, the inventive compounds unexpectedly showed enhanced antibacterial activity against bacteria of the genus Acinetobacter.

Without wishing to be bound by theory, compounds of the rifamycin class other than rifabutin are not generally active against infections caused by A. baumannii. Surprisingly, the compounds set forth herein showed activity against A. baumannii that is as good as or better than activity shown by rifabutin. Also surprisingly, the compounds set forth herein showed better antibacterial activity than rifabutin in A. baumannii strains carrying a mutation in the RNA polymerase (RpoB) gene.

As shown in Example 3, Tables 2 and 3A & 3B, the compounds of the invention are effective at inhibiting growth of a broad range of bacteria including S. aureus, A. baumannii, E. faecium, E. faecalis, S. epidermidis, S. pneumoniae, S. pyogenes, H. influenzae, M. abscessus, M. tuberculosis, and M. smegmatis. Moreover, as shown in Tables 4 and 5, compounds of the invention were effective at inhibiting the growth of strains of A. baumannii carrying mutations in the RpoB gene that confer reduced activity of rifamycins such as rifampicin and/or rifabutin. For example, Table 4 demonstrates that compounds of the invention exhibited a multi-fold increase in activity compared with rifabutin and rifampicin against A. baumannii strains carrying an H535L mutation in the RpoB gene. Table 5 shows additional examples in which compounds of the invention exhibited multi-fold increases in activity against A. baumannii strains with reduced rifabutin and/or rifampicin activity.

In one aspect, the invention provides a compound or a pharmaceutical composition as described herein, for use as a medicament. In one aspect, the invention provides a compound or a pharmaceutical composition as described herein, for use in a method of preventing or treating a disease in a subject, preferably an infection, further preferably a bacterial infection.

In some embodiments, the present invention provides a method of treating or preventing a disease in a subject in need thereof, the method comprising administering to the subject an effective amount of a compound or pharmaceutical composition of the present invention. In some embodiments, the disease to be treated is an infection, further preferably a bacterial infection.

In some embodiments, the present invention provides the use of a compound or pharmaceutical composition of the present invention in the manufacture of a medicament for treating or preventing a disease in a subject in need thereof. In some embodiments, the disease to be treated is an infection, further preferably a bacterial infection.

Accordingly, in some embodiments, the inventive compounds are used to inhibit bacterial infection. In some embodiments, the infection is caused by a bacteria belonging to Acinetobacter spp., Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii.

More preferably, the bacterial infection caused by S. aureus, A. baumannii, E. faecium, E. faecalis, S. epidermidis, S. pneumoniae, S. pyogenes, H. influenzae, M. abscessus, M. tuberculosis, and/or M. smegmatis. Yet more preferably the bacterial infection caused by one or more bacterium belonging to the genus Acinetobacter. Yet more preferably the bacterial infection is caused by A. baumannii. Even more preferably the bacterial infection is caused by A. baumannii that carries one or more resistance mechanisms against rifamycins such as rifabutin and/or rifampicin.

In some preferred embodiments, the inventive compounds are used to treat a bacterial infection caused by a strain of bacteria that carries one or more resistance genes or resistance mechanisms against currently available antibiotics (e.g., rifampicin and/or rifabutin). In some preferred embodiments, the infection is caused by a bacteria of the species A. baumannii, wherein the A. baumannii has a mutation in the RNA polymerase B (RpoB) gene, preferably a mutation that confers reduced activity of an antibiotic, preferably an antibiotic of the rifamycin class, more preferably rifabutin and/or rifampicin. In some preferred embodiments the RpoB mutation is selected from I581M; H535Q, S521T; S583L; N527D; H535C; H535L; L542F; H535N; and combinations thereof. In some preferred embodiments, the mutation in RpoB is H535L.

### Pharmaceutical Compositions

In one aspect, the invention provides a pharmaceutical composition comprising at least one compound according to any of the preceding claims, or a pharmaceutically acceptable salt, tautomer, solvate or hydrate thereof, and a pharmaceutically acceptable excipient.

Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

In some embodiments the disclosed compounds are also formulated as a suppository that is prepared from fatty emulsions or suspensions; using polyalkylene glycols such as propylene glycol, as the carrier.

In some embodiments the disclosed compounds are administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the disclosure relates to a pharmaceutical composition comprising a compound of the present disclosure and a pharmaceutically acceptable carrier. The pharmaceutically acceptable carrier can further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

The inventive compounds and pharmaceutical compositions may be administered by any suitable route, depending on the nature of the disease or disorder to be treated, e.g. orally (as syrups, tablets, capsules, lozenges, controlled-release preparations, fast-dissolving preparations, lozenges, etc.); topically (as creams, ointments, lotions, nasal sprays or aerosols, etc.); by injection (subcutaneous, intradermic, intramuscular, intravenous, etc.) or by inhalation (as a dry powder, a solution, a dispersion, etc.).

### EQUIVALENTS

While the present technology has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

### Examples

The invention is now illustrated by way of the following non-limiting examples. While particular embodiments of the invention are described below, a skilled person will appreciate that various changes and modifications can be made. References to preparations carried out in a similar manner to, or by the general method of, other preparations, may encompass variations in routine parameters such as time, temperature, workup conditions, minor changes in reagents amounts, and the like. These alternatives for the synthesis of the derivatives are within the scope of this invention.

### Abbreviations

The following list provides definitions of certain abbreviations and symbols as used herein. It will be appreciated that the list is not exhaustive, but the meaning of those abbreviations and symbols not herein below defined will be readily apparent to those skilled in the art. In describing the invention, chemical elements are identified in accordance with the Periodic Table of the Elements.
ACN acetonitrile
AcOH acetic acid
aq. aqueous
Ar argon
DCM dichloromethane
DMF dimethylformamide
eq. equivalent
EtOAc ethyl acetate
EtOH ethanol
Et₂O diethylether
h hour(s)
HPLC high performance liquid chromatography
i-PrOH isopropanol
LC liquid chromatography
M molar
min minutes
MIC minimum inhibitory concentration
MS mass spectroscopy
NMR nuclear magnetic resonance spectroscopy
rt room temperature
TR Retention time
sat. saturated

### Analytical Methods

Unless specified otherwise, the purity and identity of intermediate or example compounds were assessed by HLPC-MS as set forth below.

HPLC Analysis: Reaction monitoring and compound purity determination were performed by liquid chromatography/mass spectrometry (LC/MS). The HPLC system was a Waters 2695 LC with a photodiode array detector Waters 996; and an XBridge C18 column (3.5 μm particle size, dimensions 50 mm×4.6 mm). The mobile phases were: phase A (H₂O/ammonium formate, pH 3.75) and phase B (CH₃CN+5% H₂O/ammonium formate, pH 3.75) and compounds were eluted according to the following method:

| Time (min) | Phase A | Phase B | Flow (mL/min) |
|---|---|---|---|
| 0.00 | 100.0 | 0.0 | 2.00 |
| 0.25 | 100.0 | 0.0 | 2.00 |
| 3.50 | 0.0 | 100.0 | 2.00 |
| 4.00 | 0.0 | 100.0 | 2.00 |
| 4.05 | 100.0 | 0.0 | 2.00 |
| 5.00 | 100.0 | 0.0 | 2.00 |

Mass Spectrometry Analysis: The mass spectrometer was a Waters Alliance Micromass ZQ 2000 using electrospray ionization (polarity: negative and positive). Percent purity of compounds was determined by reversed phase HPLC using UV detection (254 nm). Structures were confirmed by MS using electro spray ionization positive (ESI+) method and reported as M+H, referring to the protonated molecular ion.

NMR Analysis: NMR spectra were recorded on a Bruker 500 MHz spectrometer with a TXI probe. Chemical shifts are given in parts per million (ppm). The assignments were made using one-dimensional (1D)¹H and ¹³C spectra and two-dimensional (2D) HSQC, HMBC and COSY spectra. Stereochemical confirmation was performed by ROESY experiment.

Compound Synthesis: All starting materials were either purchased from commercial sources or prepared according to published procedures. Reagents were purchased from commercial sources and used without further purification. The starting material 3-amino-4-deoxy-4-imino-rifamycin S was purchased from commercial sources.

Unless otherwise described, flash column chromatography purification was performed on prepacked columns (Grace Resolv™ flash cartridges, from Grace®, or FlashPure, from BUCHI) using PuriFlash430 system from Interchim. The mobile phase used was CH₃Cl/NH₃ in MeOH (1N) from 100/0 to 95/5. Preparative thin-layer chromatography (TLC) was performed using glass plates (20×20 cm) of silica gel (silica gel 60 with fluorescent indicator UV254, thickness 1 mm or 2 mm, from MACHEREY-NAGEL). The mobile phase used was cyclohexane/DCM/MeOH (3/6/1).

Antibacterial Activity: For examples below in which the corresponding starting ketone does not present an axis of symmetry, the condensation reaction can lead to the formation of multiple (e.g., two or four) diastereoisomers of the 3,4-spiro-rifamycin derivatives. The antibacterial activity of the diastereoisomeric compounds were either tested as a mixture (e.g, “A+B”), or isomers were separated by preparative TLC or flash chromatography and tested separately.

### Example 1: Synthesis and Isolation of Compounds 13A and 13B

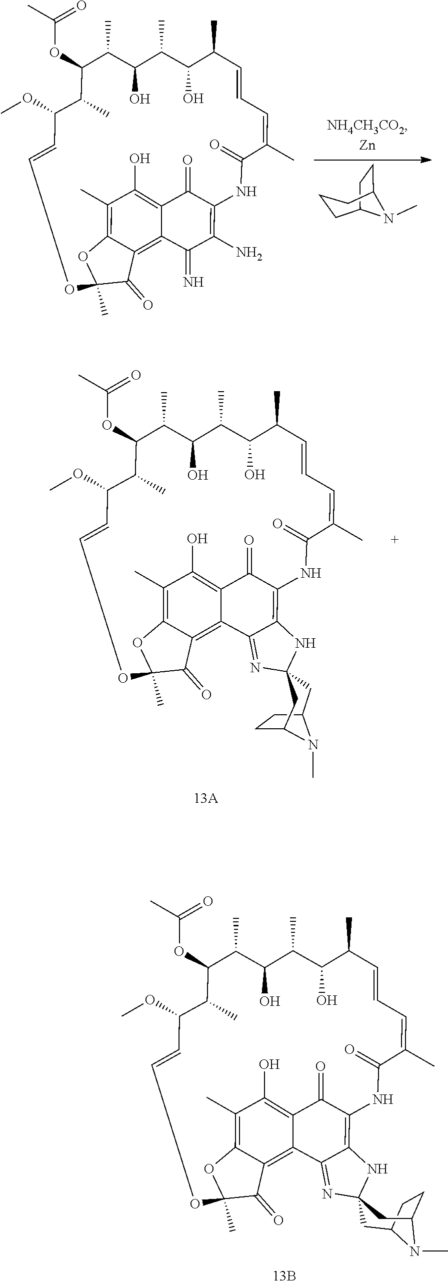

To a suspension of ammonium acetate (136 mg, 1.76 mmol, 2.5 eq) and zinc (115 mg, 1.76 mmol, 2.5 eq) in THF (5 mL) was added 3-Amino-4-Imino-Rifamycin S (500 mg, 0.70 mmol, 1 eq), acetic acid (807 μL, 14.10 mmol, 20 eq) and tropinone hydrochloride hydrate (245 mg, 1.76 mmol, 2.5 eq). The reaction was stirred at 40° C. After 1 h, the mixture was filtered over sintered glass and the THF was evaporated. The obtained oil was dissolved in EtOAc (50 mL) and washed with an aqueous solution of HCl (0.5N, 3×50 mL), a saturated solution of NaHCO₃ (3×50 mL) and brine (50 mL). The organic layer was dried over MgSO₄ and evaporated under vacuum. The obtained solid was purified by flash chromatography (CH₃Cl/NH₃ in MeOH (1N): 100/0 to 94/6) to give two products: 13A, as a purple solid (72 mg, yield=12.3%, purity (254 nm)=99%) and 13B, as a purple solid (28 mg, yield=4.7%, purity (254 nm)=99%).

The compounds shown in Table 1 were prepared according to Example 1. The compounds comprise a 3,4 spiro-rifamycin core as shown in the general structure below, and the substitution pattern shown in Table 1.

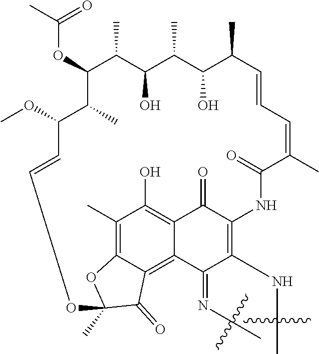

TABLE 1
Compounds Prepared According to Example 1.

| | | MW | T_{R} | ESI+ |
|---|---|---|---|---|
| Compound | Structure | (g/mol) | (min) | [M + H]+ |
| 1 (A + B) | 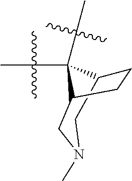 | 830.41 | 2.60 | 831.50 |
| | 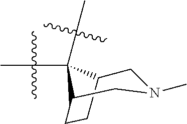 | | | |
| 2 (A + B) | 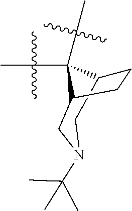 | 872.45 | 2.80 | 873.57 |
| | 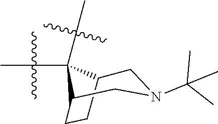 | | | |
| 3A¹ | 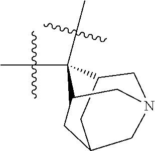 | 842.41 | 2.65 | 843.52 |
| 3B¹ | 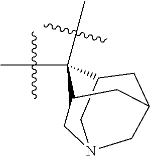 | 842.41 | 2.65 | 843.52 |
| 4 | 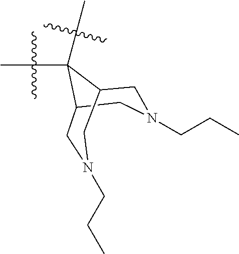 [SMILES: *C1(*)C2CN(CCC)CC1CN(CCC)C2] | 915.49 | 2.97 | 916.58 |
| 5 (A + B) | 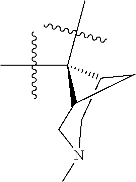 | 816.39 | 2.88 | 817.46 |
| | 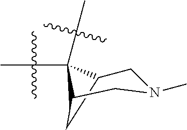 | | | |
| 6 (A + B)² | 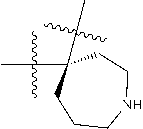 | 804.39 | 2.47 | 805.37 |
| | 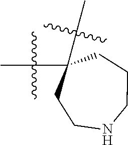 | | | |
| 7 (A + B)² | 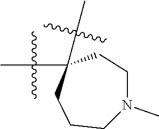 | 818.41 | 2.52 | 819.42 |
| | 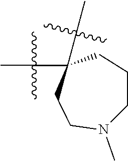 | | | |
| 8 (A, B, C, D) | 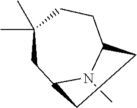 | 844.42 | 2.63 | 845.54 |
| | 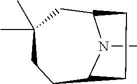 | | | |
| | 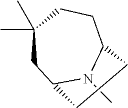 | | | |
| | 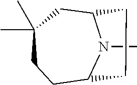 | | | |
| 9 (A + B) | 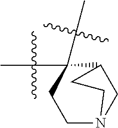 | 816.39 | 2.48 | 817.33 |
| | 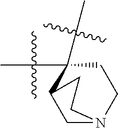 | | | |
| 10 (A + B) | 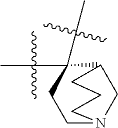 | 830.41 | 2.48 | 831.37 |
| | 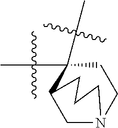 | | | |
| 11 (A + B) | 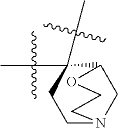 | 842.41 | 2.53 | 833.46 |
| | 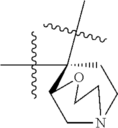 | | | |
| 12A¹ | 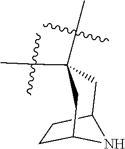 | 816.39 | 2.58 | 817.33 |
| 12B¹ | 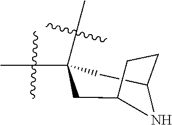 | 816.39 | 2.58 | 817.33 |
| 13A¹ | 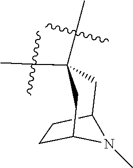 | 830.41 | 2.62 | 831.37 |
| 13B¹ | 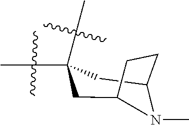 | 830.41 | 2.62 | 831.37 |
| 14A¹ | 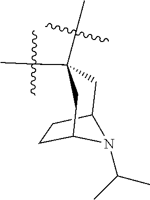 | 858.44 | 2.68 | 859.59 |
| 14B¹ | 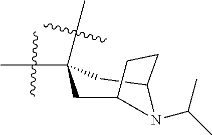 | 858.44 | 2.68 | 859.59 |
| 15 (A + B) | 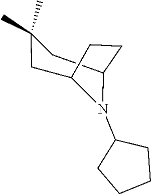 | 884.45 | 2.75 | 885.60 |
| | 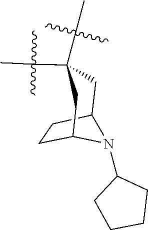 | | | |
| 16 (A + B) | 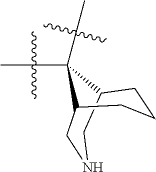 | 830.41 | 2.68 | 831.56 |
| | 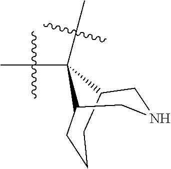 | | | |
| 17 | 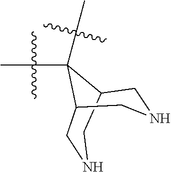 [SMILES: *C1(*)C2CNCC1CNC2] | 831.40 | 2.57 | 832.57 |
| 18 | 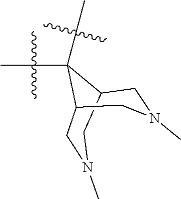 [SMILES: *C1(*)C2CN(C)CC1CN(C)C2] | 859.43 | 2.70 | 860.60 |
| 19 | 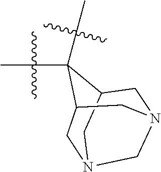 [SMILES: *C1(*)C2CN3CC1CN(C2)C3] | 843.40 | 2.49 | 844.60 |
| 20A¹ | 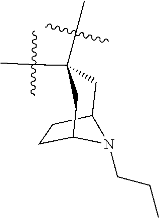 | 858.44 | 2.80 | 859.59 |
| 20B¹ | 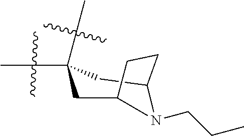 | 858.44 | 2.75 | 859.59 |

¹: Configuration attributed by NMR (¹H, ¹³C, 2D and ROESY)
²: 6A and 7A are the least polar diastereoisomer in TLC using cyclohexane/DCM/MeOH 3/6/1 as eluent, otherwise the definitive configuration has not been attributed for isomers shown in the Table.

### Example 2: Stereochemical Elucidation of Compounds 13A and 13B

The structural characterization of the new rifabutin analogs 13A and 13B was achieved using 1D and 2D NMR spectroscopic experiments through the analysis of their HSQC and HMBC spectra. Atom numbering is shown in the structure below:

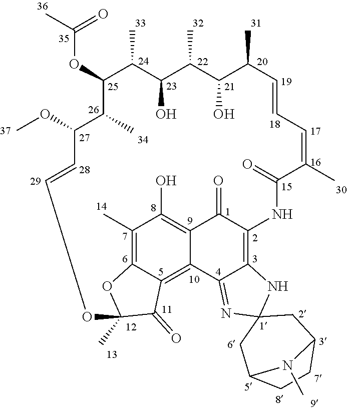

1D Experiment: ¹H NMR & ¹³C NMR

### Compound 13A

¹H NMR (500 MHz, CDCl₃): −0.04 (d, J=7.08 Hz, 3H), 0.60 (d, J=6.95 Hz, 3H), 0.82 (d, J=7.06 Hz, 3H), 1.02 (d, J=7.06 Hz, 3H), 1.32-1.39 (m, 2H), 1.60 (d, J=13.86 Hz, 1H), 1.73-1.76 (m, 4H), 1.83 (m, 1H), 1.99 (s, 3H), 2.01 (s, 3H), 2.20-2.25 (m, 2H), 2.32 (s, 3H), 2.36 (m, 1H), 2.51 (s, 3H), 2.67 (m, 2H), 2.76 (m, 2H), 2.99 (d, J=9.94 Hz, 1H), 3.08 (s, 3H), 3.31 (dd, J=7.90 Hz, J=2.76 Hz, 1H), 3.36 (bs, 1H), 3.43-3.47 (m, 2H), 3.54 (bs, 1H), 3.64 (d, J=9.94 Hz, 1H), 4.73 (d, J=10.73 Hz, 1H), 5.20 (dd, J=12.69 Hz, J=7.97 Hz, 1H), 5.94 (dd, J=15.69 Hz, J=6.50 Hz, 1H), 6.18 (d, J=12.56 Hz, 1H), 6.21 (d, J=10.35 Hz, 1H), 6.31 (dd, J=15.64 Hz, J=10.18 Hz, 1H), 8.09 (s, 1H), 14.75 (s, 1H) (chemical shifts in ppm).

¹³C NMR (500 MHz, CDCl₃): 7.7, 8.9, 11.3, 11.4, 17.4, 20.4, 21.2, 22.0, 25.3, 25.5, 27.8, 33.1, 37.6, 37.8, 38.6, 39.2, 39.6, 39.8, 56.9, 70.6, 72.5, 73.0, 81.2, 91.0, 104.6, 107.3, 108.9, 111.8, 114.9, 115.7, 124.3, 124.9, 131.4, 133.2, 140.7, 141.3, 144.5, 155.5, 168.2, 168.3, 171.5, 172.1, 181.6, 193.2 (chemical shifts in ppm).

### Compound 13B

¹H NMR (500 MHz, CDCl₃): −0.15 (d, J=7.09 Hz, 3H), 0.53 (d, J=6.94 Hz, 3H), 0.77 (d, J=6.94 Hz, 3H), 0.96 (d, J=7.01 Hz, 3H), 1.18 (m, 1H), 1.38 (m, 1H), 1.47 (d, J=14.65 Hz, 1H), 1.66-1.70 (m, 5H), 1.95 (s, 3H), 1.99 (s, 3H), 2.02 (m, 1H), 2.14 (m, 1H), 2.25 (s, 3H), 2.32-2.38 (m, 3H), 2.62 (s, 3H), 2.93 (d, J=9.90 Hz, 1H), 2.99-3.05 (m, 6H), 3.31 (dd, J=6.99 Hz, J=2.1 Hz, 1H), 3.53-3.61 (m, 4H), 4.68 (d, J=10.49 Hz, 1H), 5.05 (dd, J=12.43 Hz, J=7.22 Hz, 1H), 5.94 (dd, J=15.93 Hz, J=6.77 Hz, 1H), 6.07 (d, J=12.62 Hz, 1H), 6.18 (d, J=10.49 Hz, 1H), 6.29 (dd, J=15.73 Hz, J=10.48 Hz, 1H), 8.16 (s, 1H), 9.70 (s, 1H), 14.56 (s, 1H) (chemical shifts in ppm).

¹³C NMR (500 MHz, CDCl₃): 7.6, 8.8, 10.8, 11.2, 17.4, 20.2, 21.0, 21.8, 25.4, 25.6, 33.0, 35.9, 36.8, 37.5, 37.7, 37.9, 56.9, 58.6, 58.7, 73.0, 73.1, 76.8, 80.4, 92.0, 104.3, 107.2, 109.0, 111.5, 114.5, 116.0, 123.9, 124.6, 131.3, 132.8, 141.0, 142.2, 144.0, 155.2, 168.1, 168.9, 171.4, 172.2, 180.9, 192.5 (chemical shifts in ppm).

### 2D Experiment—ROESY NMR (Compound 13B)

The stereochemistry of the diastereoisomers was assigned by studying the ROESY spectra. ROESY analysis show a correlation between NH-3 and H-7′, H-8′, H-2′ and H-6′.

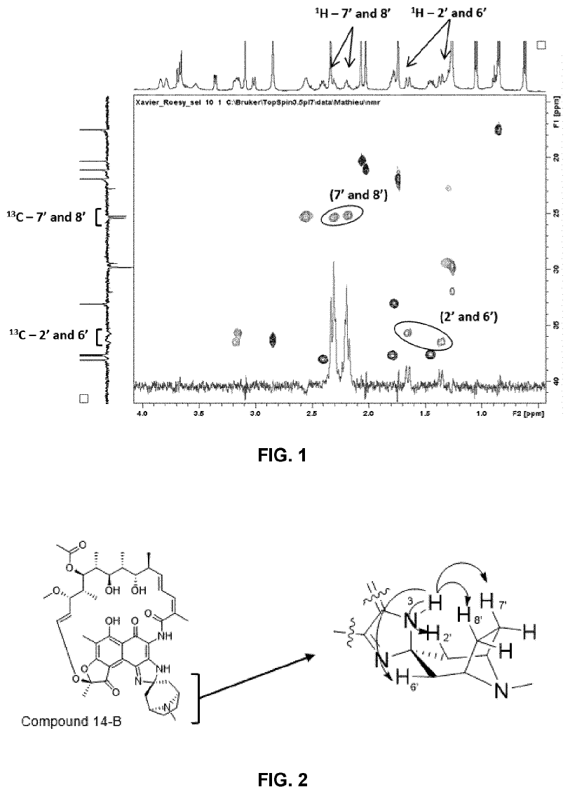

For the minor isomer 13B, the proposed disposition of the ethylenic bridge was supported by NOESY correlation peaks between the amine proton NH-3 and the protons H-7′ and H-8′ of the ethylenic part of the tropane moiety as shown in FIG. 1. This observation leads to the conclusion that the compound 13B is the diastereomer with the ethylenic moiety oriented toward the NH-3, as depicted in FIG. 2.

### Example 3: Antibacterial Activity

The in vitro antibacterial activity of the compounds disclosed was measured according to the following protocols:

MIC values were determined by broth microdilution method according to the CLSI guidelines. Unless otherwise mentioned, MIC against Acinetobacter baumannii was performed in RPMI medium supplemented with 10% FCS. MIC against Streptococcus pneumoniae and Streptococcus pyogenes was performed in cation adjusted Mueller Hinton broth supplemented with 5% laked horse blood. MIC against Hemophilus influenzae was performed in Hemophilus test medium broth. MIC against Mycobacterium abscessus, Mycobacterium tuberculosis and Mycobacterium smegmatis was performed in Middlebrook 7H9 broth supplemented with Middlebrook ADC growth supplement. All other MIC were performed in standard cation adjusted Mueller Hinton broth.

The following procedure applies to all species tested except M. tuberculosis and M. smegmatis. From an overnight culture plate, cells were resuspended in 0.9% (w/v) saline solution and bacterial inoculum was prepared in the respective testing medium with 5×10⁵ CFU/mL. The appropriate volumes of a 10 mg/mL compound solution were directly dispensed in the 96-well assay plate using a digital dispenser to create two-fold dilution series from 32 to 0.002 μg/mL final concentration. 100 μL of bacterial suspension was finally added to the compounds. Plates were covered and incubated without shaking at 35° C. for 20 hours. Every experiment contained an antibiotic as quality control. MIC was determined visually as the lowest concentration of a compound that prevents visible growth of the bacteria and the plates were scanned for documentation.

A similar procedure was used for M. tuberculosis and M. smegmatis, except that the inoculum was prepared from an exponentially growing culture and set to an OD₆₀₀ of 0.02 and 0.002 for M. tuberculosis and M. smegmatis, respectively. The MIC was determined at 90% growth inhibition using a GFP reporter after 5 days incubation for M. tuberculosis and using the BacTiter-Glo kit (Promega) after 20 hours incubation for M. smegmatis.

The results are shown below in the following Tables.

TABLE 2
In vitro antibacterial activity of compounds.

| MIC (mg/mL) | S. aureus | A. baumannii |
|---|---|---|
| Rifabutin | 0.016 | ≤0.002 |
| 1 (A + B) | 0.016 | 0.25 |
| 2 (A + B) | 0.008 | 2 |
| 3A | 0.008 | 0.25 |
| 3B | 0.008 | ≤0.002 |
| 4 | 0.016 | 0.03 |
| 5 (A + B) | 0.125 | 8 |
| 6A | 0.016 | 0.125 |
| 6B | 0.016 | 0.125 |
| 7A | 0.008 | 0.004 |
| 7B | 0.008 | 0.125 |
| 8 (A + B + C + D) | 0.03 | 0.008 |
| 9 (A + B) | 0.016 | 0.25 |
| 10 (A + B) | 0.008 | 0.06 |
| 11 (A + B) | 0.004 | 4 |
| 12A | 0.06 | 0.06 |
| 12B | 0.016 | ≤0.002 |
| 13A | 0.03 | 0.03 |
| 13B | 0.016 | ≤0.002 |
| 14A | 0.016 | 0.25 |
| 14B | 0.008 | 0.016 |
| 15 (A + B) | 0.03 | 0.25 |
| 16 (A + B) | 0.004 | ≤0.002 |
| 17 | 0.008 | 0.5 |
| 18 | 0.016 | 0.008 |
| 19 | 0.004 | 1 |
| 20A | 0.008 | 0.25 |
| 20B | 0.004 | ≤0.002 |

Compounds 3B, 7A, 8(A+B+C+D), 12B, 13B, 16 (A+B), 18 and 20B were tested against the following other representative species: Enterococcus faecium (strain NCTC 12204), Enterococcus faecalis (strain ATCC 51575), Staphylococcus epidermidis (strain ATCC 12228), S. pneumoniae (strain ATCC 49619), S. pyogenes (strain IHMA 1117021), H. influenzae (strain ATCC 49247), M. abscessus (strain ATCC 19977), M. tuberculosis (H37Rv strain ATCC 27294) and M. smegmatis (strain ATCC 700084). The results are given in Table 3A and Table 3B.

TABLE 3A
In vitro antibacterial activity of selected
compounds against listed species.

| MIC (mg/mL) | E. faecium | E. faecalis | S. epidermidis | S. pneumoniae |
|---|---|---|---|---|
| Rifampicin | 0.5 | 1 | ≤0.002 | 0.016 |
| Rifabutin | 0.125 | 4 | 0.004 | ≤0.002 |
| 3B | 0.03 | 0.5 | ≤0.002 | ≤0.002 |
| 7A | 0.06 | 0.5 | ≤0.002 | ≤0.002 |
| 8 (A + B + | 0.125 | 2 | ≤0.002 | ≤0.002 |
| C + D) | | | | |
| 12B | 0.03 | 0.125 | ≤0.002 | ≤0.002 |
| 13B | 0.125 | 0.5 | ≤0.002 | ≤0.002 |
| 16(A + B) | 0.125 | 0.5 | ≤0.002 | ≤0.002 |
| 18 | 0.125 | 2 | ≤0.002 | ≤0.002 |
| 20B | 0.5 | 1 | ≤0.002 | ≤0.002 |

TABLE 3B
In vitro antibacterial activity of selected compounds against listed species.

| MIC (mg/mL) | S. pyogenes | H. influenzae | M. abscessus | M. tuberculosis | M. smegmatis |
|---|---|---|---|---|---|
| Rifampicin | 0.03 | 0.25 | >32 | 0.0041 | 1.12 |
| Rifabutin | 0.004 | 0.5 | 8 | 0.0035 | 0.24 |
| 3B | ≤0.002 | 0.125 | 8 | 0.010 | 0.65 |
| 7A | ≤0.002 | 0.06 | 16 | 0.010 | 0.54 |
| 8 (A + B + C + D) | 0.016 | 0.125 | 32 | 0.010 | 1.19 |
| 12B | ≤0.002 | 0.06 | 8 | 0.010 | 0.32 |
| 13B | ≤0.002 | 0.125 | 8 | 0.010 | 0.30 |
| 16(A + B) | ≤0.002 | 0.06 | 4 | ND | 0.33 |
| 18 | ≤0.002 | 0.125 | 32 | 0.010 | 1.11 |
| 20B | ≤0.002 | 0.25 | 8 | 0.010 | 0.29 |

The same compounds were tested against representative A. baumannii clinical isolates with mutations in RpoB known to confer reduced rifabutin activity (Trebosc et al. J. Antimicrob. Chemother. 2020; 75(12):3552-3562). The results are given in Table 4.

TABLE 4
In vitro antibacterial activity of selected compounds against
A. baumannii strains carrying RpoB mutations.

A. baumannii MIC (mg/L) in RPMI + FCS

| | | RpoB | RpoB | RpoB |
|---|---|---|---|---|
| Compound | WT | H535Q | H535C | H535L |
| Rifampicin^{a} | 8 | >32 | >32 | >32 |
| Rifabutin | ≤0.002 | 0.004 | 0.03 | >32 |
| 3B | ≤0.002 | ≤0.002 | ≤0.002 | 0.016 |
| 7A | 0.004 | 0.016 | 0.03 | 1 |
| 8(A + B) | 0.008 | 0.06 | 0.125 | 1 |
| 12B | ≤0.002 | ≤0.002 | ≤0.002 | 0.03 |
| 13B | ≤0.002 | ≤0.002 | ≤0.002 | 0.03 |
| 16(A + B) | ≤0.002 | 0.008 | 0.016 | 1 |
| 18 | 0.008 | 0.03 | 0.25 | 4 |
| 20B | ≤0.002 | 0.004 | 0.008 | 0.5 |

^{a}Rifampicin MIC determined in cation-adjusted Mueller Hinton broth

The in vitro activity of compounds 3B, 12B and 13B was further tested against all known A. baumannii clinical isolates carrying an RpoB mutation (n=20) from a collection of 353 A. baumannii strains. The results are given in Table 5.

TABLE 5
In vitro antibacterial activity (MIC mg/L) against
A. baumannii isolates with various RpoB mutations.

| | RpoB mutation | | | | | |
|---|---|---|---|---|---|---|
| Compound | I581M | H535Q | H535Q | H535Q | H535Q/S521T | H535Q/S521T |
| Rifampicin^{a} | >32 | >32 | >32 | >32 | ND | ND |
| Rifabutin | ≤0.002 | 0.03 | >32 | 16 | 32 | 16 |
| 3B | ≤0.002 | ≤0.002 | 2 | 1 | 1 | 0.5 |
| 12B | 0.004 | ≤0.002 | 4 | 1 | 0.5 | 1 |
| 13B | ≤0.002 | ≤0.002 | 4 | 2 | 0.5 | 1 |

| | RpoB mutation | | | | | |
|---|---|---|---|---|---|---|
| Compound | H535C | H535C | H535C | H535C | H535L | H535L |
| Rifampicin^{a} | ND | >32 | >32 | >32 | >32 | >32 |
| Rifabutin | 0.06 | 0.016 | 0.25 | >32 | 0.06 | 0.008 |
| 3B | ≤0.002 | ≤0.002 | 0.004 | 0.008 | 0.008 | ≤0.002 |
| 12B | ≤0.002 | 0.002 | 0.004 | 0.016 | 0.004 | ≤0.002 |
| 13B | ≤0.002 | 0.002 | 0.004 | 0.016 | 0.004 | ≤0.002 |

| | RpoB mutation | | | | | | |
|---|---|---|---|---|---|---|---|
| Compound | H535L | L542F | L542F | S583L | N527D | H535L | H535L |
| Rifampicin^{a} | >32 | 8 | 8 | >32 | >32 | >32 | >32 |
| Rifabutin | >32 | 0.004 | 1 | 0.03 | 0.03 | >32 | >32 |
| 3B | 0.125 | ≤0.002 | 0.125 | ≤0.002 | ≤0.002 | 0.016 | 0.016 |
| 12B | 0.125 | 0.002 | 0.125 | 0.004 | ≤0.002 | 0.03 | 0.06 |
| 13B | 0.25 | 0.002 | 0.125 | 0.004 | ≤0.002 | 0.03 | 0.03 |

^{a}Rifampicin MIC determined in cation-adjusted Mueller Hinton broth
ND: not determined

1. A compound of Formula I or a pharmaceutically acceptable salt, tautomer, solvate, hydrate, enantiomer or diastereomer thereof: 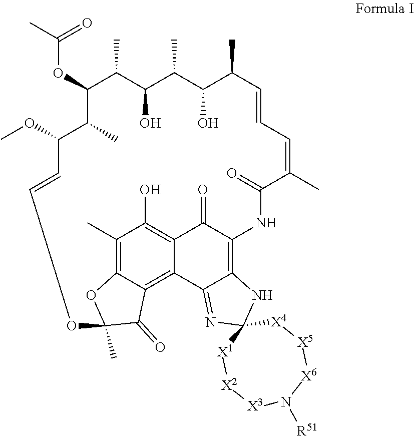
wherein:
X¹ is —CR¹R^{1A}—;
X² is independently, at each occurrence, selected from —CR²R^{2A}—, —NR^{2B}—, —O—, and a chemical bond;
X³ is independently, at each occurrence, selected from —CR³R^{3A}—, —NR^{3B}, —O—, and a chemical bond;
X⁴ is —CR⁴R^{4A}—;
X⁵ is independently, at each occurrence, selected from —CR⁵R^{5A}—, —NR^{5B}—, —O—, and a chemical bond;
X⁶ is independently, at each occurrence, selected from —CR⁶R^{6A}—, —NR^{6B}—, —O—, and a chemical bond;
R⁵¹ is independently, at each occurrence, selected from —H, —C₁-C₆ alkyl, and —C₃-C₈ cycloalkyl;
R¹, R^{1A}, R², R^{2A}, R³, R^{3A}, R⁴, R^{4A}, R⁵, R^{5A}, R⁶ and R^{6A} are independently, at each occurrence, selected from —H, —C₁-C₆ alkyl, and —C₁-C₆ alkoxy;
R^{2B}, R^{3B}, R^{5B} and R^{6B} are independently, at each occurrence, selected from —H and —C₁-C₆ alkyl; or
any of R¹, R^{1A}, R², R^{2A}, R^{2B}, R³, R^{5A}, R^{3B}, R⁴, R^{4A}, R⁵, R^{5A}, R^{5B}, R⁶, R^{6A}, R^{6B} and R⁵¹, together with the atom to which they are attached, can independently combine with any other member of R¹, R^{1A}, R², R^{2A}, R^{2B}, R³, R^{3A}, R^{3B}, R⁴, R^{4A}, R⁵, R^{5A}, R^{5B}, R⁶, R^{6A}, R^{6B} and R⁵¹ to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₆ alkyl or —C₁-C₆ alkoxy;
wherein when any of R¹, R^{1A}, R², R^{2A}, R^{2B}, R³, R^{3A}, R^{3B}, R⁴, R^{4A}, R⁵, R^{5A}, R^{5B}, R⁶, R^{6A} and R^{6B} combine to form said first bridging cycloalkyl ring or said first bridging heterocycloalkyl ring, said first bridging cycloalkyl or first bridging heterocycloalkyl can independently combine with R⁵¹ to form a second bridging 5-7 membered heterocycloalkyl, wherein said second bridging heterocycloalkyl is optionally substituted by one or more —C₁-C₆ alkyl, or —C₁-C₆ alkoxy; and
provided that at least two of R¹, R^{1A}, R², R^{2A}, R^{2B}, R³, R^{3A}, R^{3B}, R⁴, R^{4A}, R⁵, R^{5A}, R^{5B}, R⁶, R^{6A}, R^{6B} and R⁵¹ combine to form a bridging cycloalkyl or heterocycloalkyl; or no more than one of X², X³, X⁵, and X⁶ is a chemical bond.

2. The compound of claim 1, wherein:
X¹, X², X³, X⁴, X⁵, X⁶ and NR⁵¹ combine to form an azepane, wherein said azepane optionally further comprises a bridging C₄-C₈ cycloalkyl or bridging 5 to 8-membered heterocycloalkyl; or
X¹, X², X³, X⁴, X⁵, X⁶ and NR⁵¹ combine to form a piperidine further comprising a bridging C₄-C₈ cycloalkyl or bridging 5 to 8-membered heterocycloalkyl.

3. The compound of any of the preceding claims, wherein:
X¹ is CR¹R^{1A};
X² is CR²R^{2A};
X³ is independently, at each occurrence, selected from CR³R^{3A} and a chemical bond;
X⁴ is CR⁴R^{4A};
X⁵ is CR⁵R^{5A};
X⁶ is independently, at each occurrence, selected from CR⁶R^{6A} and a chemical bond;
R¹, R^{1A}, R², R^{2A}, R³, R^{3A}, R⁴, R^{4A}, R⁵, R^{5A}, R⁶ and R^{6A} are independently, at each occurrence, selected from —H, —C₁-C₆ alkyl, and —C₁-C₆ alkoxy;
R⁵¹ is independently, at each occurrence, selected from —H, —C₁-C₆ alkyl, and —C₃-C₈ cycloalkyl; or
any of R¹, R² and R³, together with the carbon atoms to which they are attached, can independently combine with any of R⁴, R⁵, R⁶, and R⁵¹ to form a bridging C₄-C₈ cycloalkyl ring or a bridging 5-8 membered heterocycloalkyl ring; or any of R⁴, R⁵, R⁶, together with the carbon atoms to which they are attached, can independently combine with R⁵¹ to form a bridging 5-8 membered heterocycloalkyl ring, wherein said heterocycloalkyl is optionally substituted by one or more —C₁-C₆ alkyl or —C₁-C₆ alkoxy;
wherein when any of R¹, R², R³, R⁴, R⁵, and R⁶ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted by one or more —C₁-C₆ alkyl, or —C₁-C₆ alkoxy; and
provided that at least two of R¹, R², R³, R⁴, R⁵, R⁶ and R⁵¹ combine to form a bridging cycloalkyl or heterocycloalkyl; or no more than one of X³ and X⁶ is a chemical bond.

4. The compound of any of the preceding claims, wherein:
X¹ is CR¹R^{1A}; X² is CR²R^{2A}; X³ is independently, at each occurrence, selected from CR³R^{3A} and a chemical bond; X⁴ is CR⁴R^{4A}; X⁵ is CR⁵R^{5A}; X⁶ is independently, at each occurrence, selected from CR⁶R^{6A} and a chemical bond; wherein
(i) R^{1A}, R², R^{2A}, R^{4A}, R⁵ and R^{5A} are —H; R³, R^{3A}, R⁶ and R^{6A} are absent or —H; R¹ and R⁴ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(ii) R^{1A}, R¹, R^{2A}, R^{4A}, R⁴ and R^{5A} are —H; R³, R^{3A}, R⁶ and R^{6A} are absent or —H; R² and R⁵ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(iii) R^{1A}, R², R^{2A}, R⁴, R^{4A}, R⁵ and R^{5A} are —H; R³, R^{3A}, R⁶ and R^{6A} are absent or —H; R¹ and R⁵¹ combine to form a bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(iv) R¹, R^{1A}, R², R^{2A}, R^{4A}, R⁵, and R^{5A} are —H; R³, R^{3A}, R⁶ and R^{6A} are absent or —H; R⁴ and R⁵¹ combine to form a bridging 5-8 membered heterocycloalkyl ring, wherein said bridging heterocycloalkyl is optionally substituted by one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; wherein
when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl, wherein each bridging cycloalkyl or heterocycloalkyl is optionally substituted with one or more —C₁-C₄ alkyl or —C₁-C₄ alkoxy, preferably —C₁-C₃ alkyl or —C₁-C₃ alkoxy, more preferably —C₁-C₂ alkyl or C₁-C₂ alkoxy; or
(v) exactly one of X³ and X⁶ is a chemical bond.

5. The compound of any of the preceding claims, wherein:
X¹ is CR¹R^{1A}; X² is CR²R^{2A}; X⁴ is CR⁴R^{4A}; X⁵ is CR⁵R^{5A};
(i) X³ and X⁶ are both chemical bonds, R^{1A}, R², R^{2A}, R^{4A}, R⁵, and R^{5A} are —H, R¹ and R⁴ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₃ alkyl or —C₁-C₃ alkoxy, preferably —C₁-C₂ alkyl or —C₁-C₂ alkoxy; or
(ii) X³ and X⁶ are both chemical bonds, R^{1A}, R¹, R^{2A}, R^{4A}, R⁴, and R^{5A} are —H, R² and R⁵ combine to form a first bridging C₄-C₈ cycloalkyl ring or a first bridging 5-8 membered heterocycloalkyl ring, wherein each bridging cycloalkyl or bridging heterocycloalkyl is optionally substituted by one or more —C₁-C₃ alkyl or —C₁-C₃ alkoxy, preferably —C₁-C₂ alkyl or —C₁-C₂ alkoxy; or
(iii) X³ and X⁶ are both chemical bonds, R^{1A}, R², R^{2A}, R^{4A}, R⁵, and R^{5A} are —H, one of R¹ and R⁴ combine with R⁵¹ to form a bridging 5-8 membered heterocycloalkyl ring, wherein said bridging heterocycloalkyl is optionally substituted by one or more —C₁-C₃ alkyl or —C₁-C₃ alkoxy, preferably —C₁-C₂ alkyl or —C₁-C₂ alkoxy; wherein
when R¹ and R⁴ or R² and R⁵ combine to form a first bridging cycloalkyl ring or first bridging heterocycloalkyl ring, the first bridging cycloalkyl or first bridging heterocycloalkyl can independently combine with R⁵¹ to form a second bridging 5-7 membered heterocycloalkyl wherein the second bridging heterocycloalkyl is optionally substituted by one or more —C₁-C₃ alkyl or —C₁-C₃ alkoxy, preferably —C₁-C₂ alkyl or —C₁-C₂ alkoxy; or
(iv) exactly one of X³ and X⁶ is a chemical bond.

6. The compound of any of the preceding claims, wherein X¹ is CR¹R^{1A}; X² is CR²R^{2A}; X⁴ is CR⁴R^{4A}; X⁵ is CR⁵R^{1A};
(i) X³ and X⁶ are both chemical bonds, R^{1A}, R², R^{2A}, R^{4A}, R⁵, and R^{5A} are —H, R¹ and R⁴ combine to form a first bridging cyclobutyl, cyclopentyl, cyclohexyl, or piperidinyl ring, wherein each cyclobutyl, cyclopentyl, cyclohexyl, or piperidinyl ring is optionally substituted with one or more —C₁-C₃ alkyl, preferably one or two —C₁-C₂ alkyl; or
(ii) X³ and X⁶ are both chemical bonds, R^{1A}, R¹, R^{2A}, R^{4A}, R⁴, and R^{1A} are —H, R² and R⁵ combine to form a first bridging cyclohexyl or cycloheptyl ring, wherein each cyclohexyl or cycloheptyl is optionally substituted with one or more —C₁-C₃ alkyl, preferably one or two —C₁-C₂ alkyl; or
(iii) X³ and X⁶ are both chemical bonds, R^{1A}, R², R^{2A}, R^{4A}, R⁵, and R^{5A} are —H, one of R¹ and R⁴ combine with R⁵¹ to form a bridging pyrrolidinyl, piperidinyl, or oxazinanyl, wherein each pyrrolidinyl, piperidinyl, or oxazinanyl is optionally substituted with one or more —C₁-C₃ alkyl, preferably one or two —C₁-C₂ alkyl; wherein
when R¹ and R⁴ or R² and R⁵ combine to form a first bridging cyclohexyl or piperidinyl, the first bridging cyclohexyl or piperidinyl can independently combine with R⁵¹ to form a second bridging piperidinyl or 1,3-diazinanyl, wherein the second bridging piperidinyl or 1,3-diazinanyl is optionally substituted by one or more —C₁-C₃ alkyl, preferably one or two —C₁-C₂ alkyl; or
(iv) exactly one of X³ and X⁶ is a chemical bond.

7. The compound of any of the preceding claims, wherein R⁵¹ is selected from —H, —C₁-C₄ alkyl, and —C₅-C₆ cycloalkyl.

8. The compound of any of the preceding claims, wherein exactly one of X³ and X⁶ is a chemical bond.

9. The compound of any of the preceding claims, wherein X³ and X⁶ are both chemical bonds.

10. The compound of claim 1, wherein the compound is of Formula IA, IB, IC, ID, or TE: 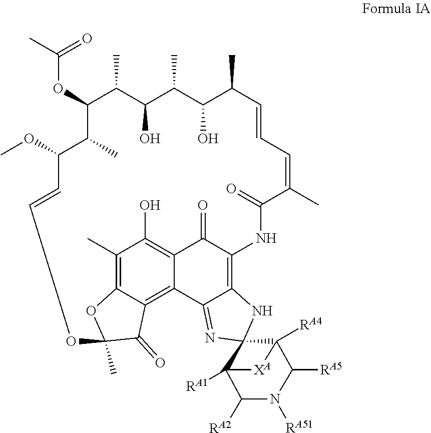
wherein:
R^{A1}, R^{A2}, R^{A4} and R^{A5} are each independently —H or —C₁-C₄ alkyl;
X^{A} is independently selected from —CH₂—, —CH₂CH₂—, —CH₂CH₂CH₂—, —CH₂OCH₂—, —CH₂NHCH₂—, or —CH₂N(C₁-C₄ alkyl)CH₂;
R^{A5} is independently —H or —C₁-C₄ alkyl; or
X^{A} and R^{A51}, together with the atoms to which they are attached, combine to form a 5 to 6-membered heterocycloalkyl; 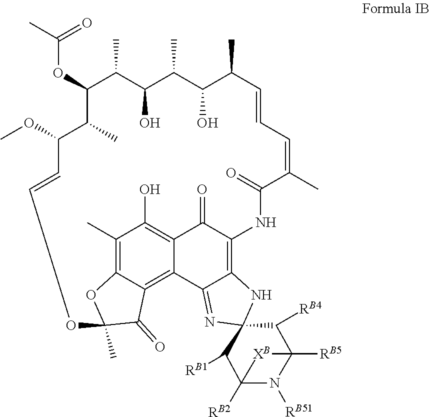
wherein:
R^{B1}, R^{B2}, R^{B4} and R^{B5} are each independently —H or —C₁-C₄ alkyl;
X^{B} is independently selected from —CH₂—, and —CH₂CH₂—;
R^{B51} is independently —H or —C₁-C₄ alkyl; 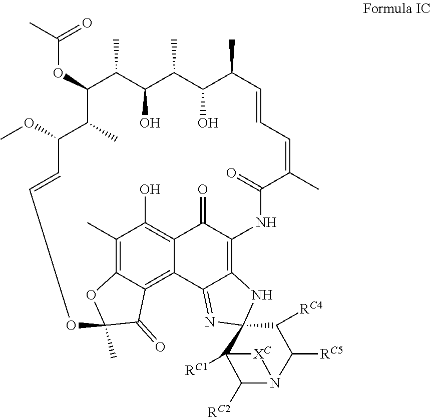
wherein:
R^{C1}, R^{C2}, R^{C4} and R^{C5} are each independently —H or —C₁-C₄ alkyl;
X° is independently selected from —CH₂CH₂—, —CH₂CH₂CH₂—, —OCH₂CH₂—, and —NHCH₂CH₂—; 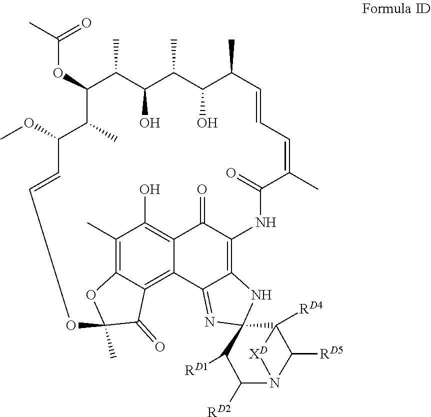
wherein:
R^{D1}, R^{D2}, R^{D4} and R^{D5} are each independently —H or —C₁-C₄ alkyl;
X^{D} is independently selected from —CH₂CH₂—, —CH₂CH₂CH₂—, —OCH₂CH₂—, and —NHCH₂CH₂—; 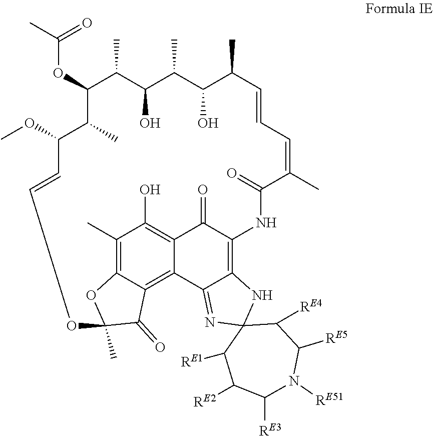
wherein:
R^{E1}, R^{E2}, and R^{E4} are each —H or C₁-C₄ alkyl; or
R^{E3} and R^{E5} are —H or combine to form a bridging cyclooctane; and;
R^{E51} is —H or —C₁-C₄ alkyl.

11. The compound of claim 1, wherein X¹, X², X³, X⁴, X⁵, X⁶ and NR⁵¹ form a structure selected from:

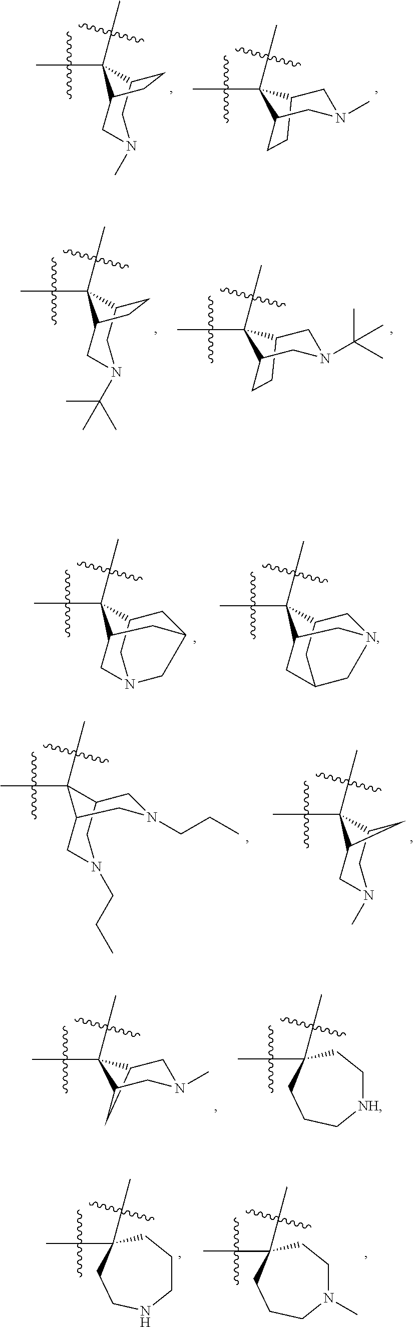

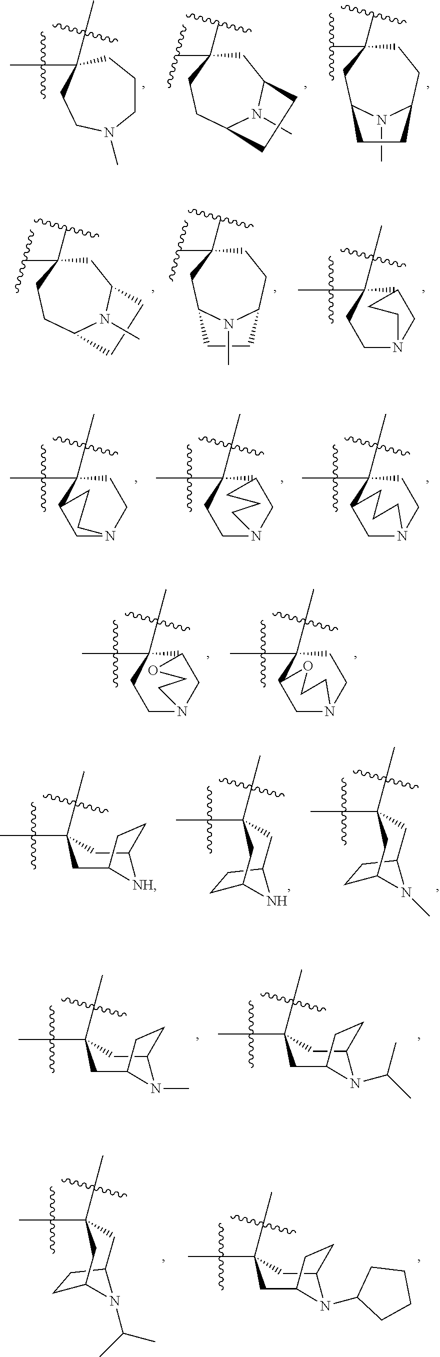

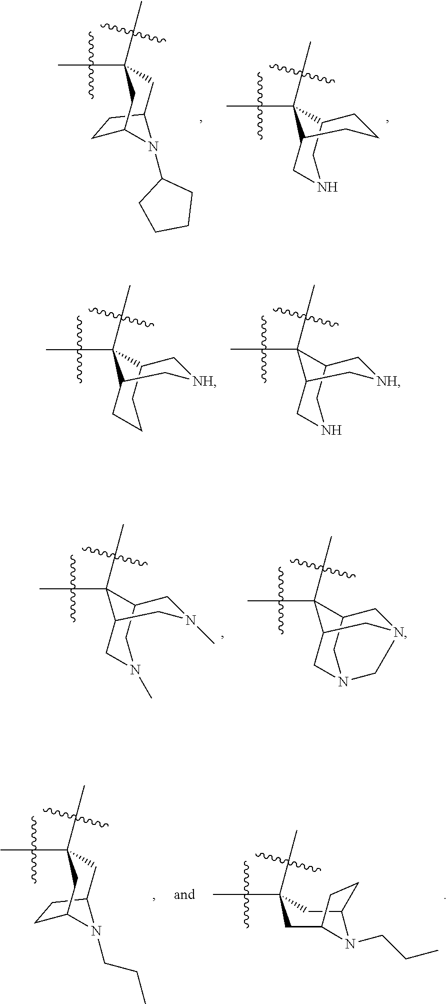

12. The compound of claim 1, selected from the group consisting of: 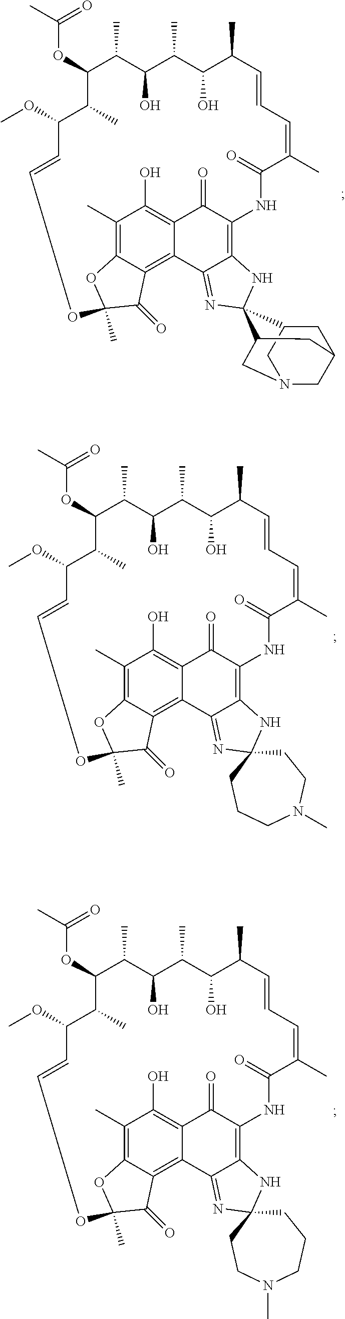 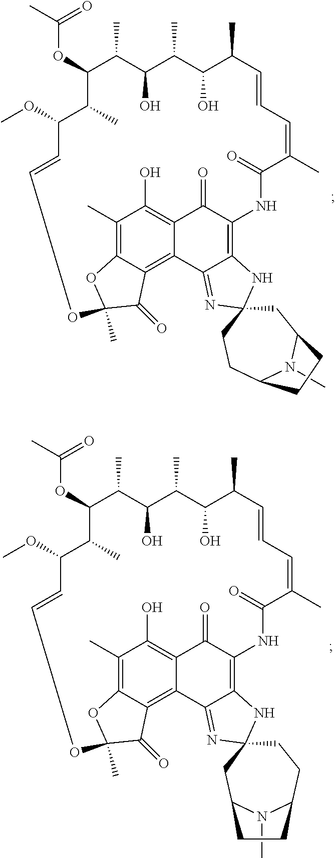 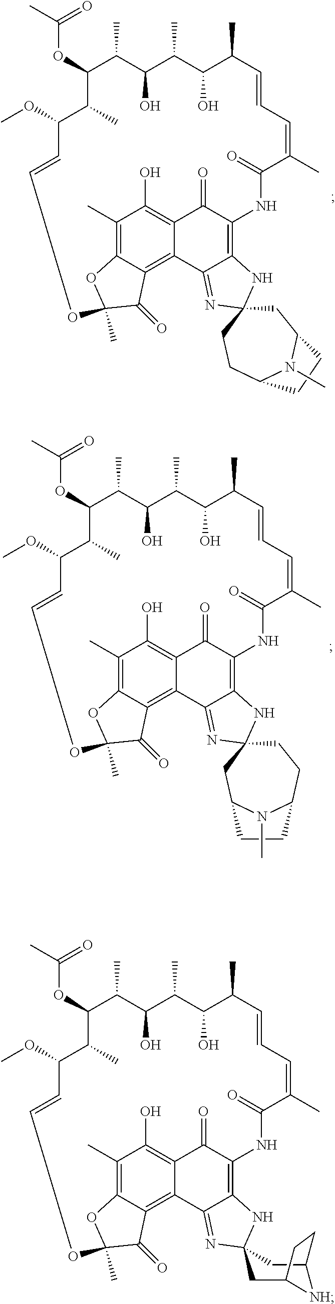 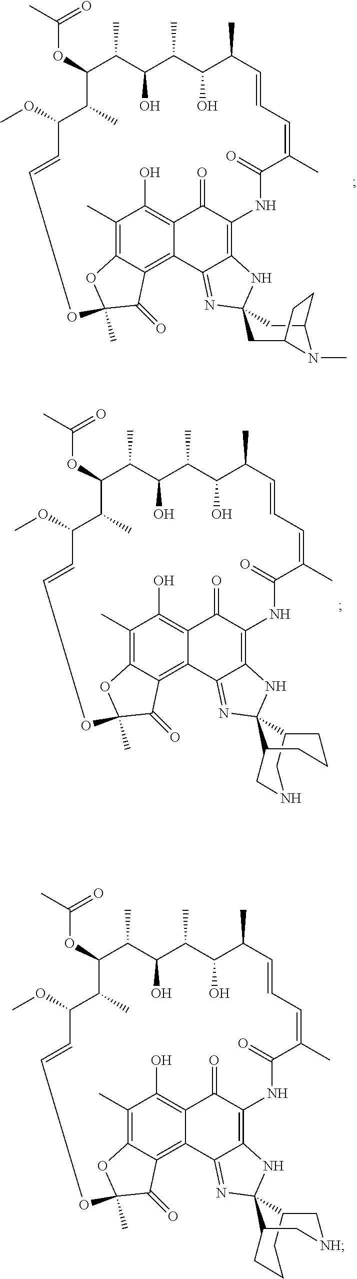 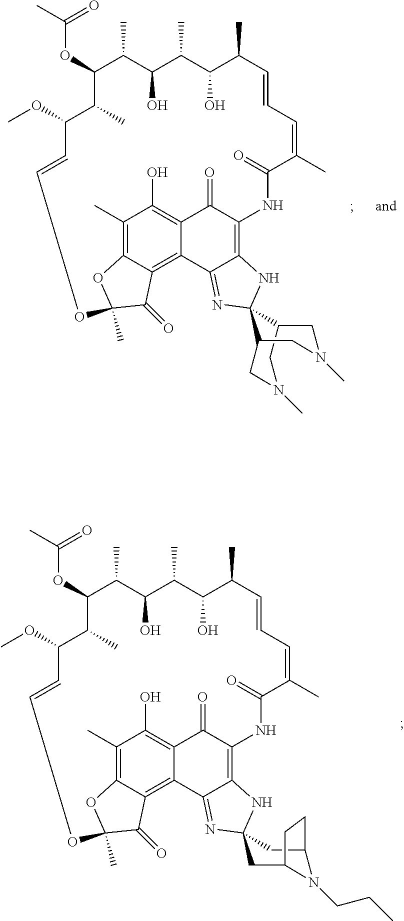
wherein preferably said compound is selected from the group consisting of:

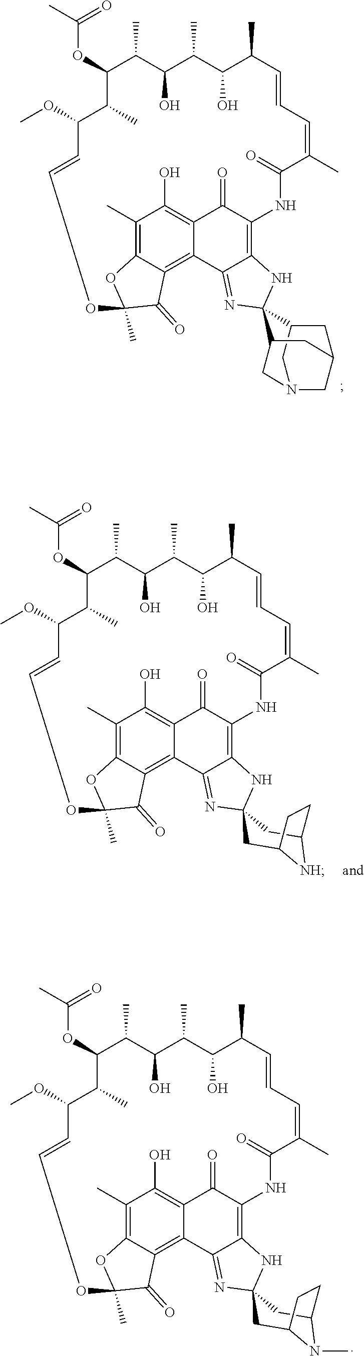

13. A pharmaceutical composition comprising at least one compound according to any of the preceding claims, or a pharmaceutically acceptable salt, tautomer, solvate or hydrate thereof, and a pharmaceutically acceptable excipient.

14. The compound according to any of claims 1-12, or a pharmaceutical composition according to claim 13, for use as a medicament, preferably for use in a method of preventing or treating a disease in a subject, wherein said disease is an infection, wherein preferably said disease is a bacterial infection.

15. The compound or pharmaceutical composition for use of claim 14, wherein said disease is a bacterial infection, wherein said bacterial infection is caused by a bacteria belonging to Acinetobacter spp., Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii; more preferably a bacterial infection caused by S. aureus, A. baumannii, E. faecium, F faecalis, S. epidermidis, S. pneumoniae, S. pyogenes, H. influenzae, M. abscessus, M. tuberculosis, and/or M. smegmatis; and wherein preferably said bacterial infection is caused by one or more bacterium belonging to the genus Acinetobacter; further preferably by A. baumannii; and even more preferably by A. baumannii that carries one or more resistance mechanisms against rifamycins such as rifabutin and/or rifampicin.
